(19) United States
(12) Patent Application Publication
(10) Pub. No.: US 2023/0295638 A1
(43) Pub. Date: Sep. 21, 2023

(54) MODULAR PLATFORM FOR PROGRAMMABLE SPATIOTEMPORAL BIOMOLECULE CLUSTERING WITH APPLICATIONS INCLUDING ENHANCED METABOLIC YIELD

(71) Applicant: The Trustees of Princeton University, Princeton, NJ (US)

(72) Inventors: Clifford BRANGWYNNE, Hopewell, NJ (US); José L. AVALOS, Princeton, NJ (US); Mackenzie Thomas WALLS, Cinnaminson, NJ (US)

(73) Assignee: The Trustees of Princeton University, Princeton, NJ (US)

(21) Appl. No.: 17/928,028

(22) PCT Filed: May 27, 2021
(86) PCT No.: PCT/US2021/034570

§ 371 Date: Nov. 28, 2022

Related U.S. Application Data
(60) Provisional application No. 63/031,388, filed on May 28, 2020.

(51) Int. Cl. C12N 15/62; C12Q 1/02; C12N 9/10; C12N 9/88; C07K 14/395

(52) U.S. Cl. CPC C12N 15/62; C12Q 1/025; C12N 9/1014; C12Y 202/01001; C12Y 202/01002; C12N 9/1085; C12Y 205/01054; C12N 9/88; C12Y 402/01011; C07K 14/395; C07K 2319/735; C07K 2319/60

(57) ABSTRACT

Provided herein are programmable condensate protein systems and nucleic acid constructs encoding the same. The protein system enables modular targeting of proteins of interest. Protein-peptide interaction domains (PPIDs) are incorporated to functionalize engineered condensates with the attributes of the recruited protein, resulting in a modular system that allows for diverse facile and reprogrammable applications, including in enzyme clustering of metabolic pathways. Colocalizing specific metabolic enzymes in these condensates results in functionalized organelles with which can be used to manipulate the output of engineered metabolic pathways for the production of a pharmaceutical precursor.

### CROSS REFERENCE TO RELATED APPLICATIONS

This application claims the benefit of U.S. Provisional Application No. 63/031,388, filed May 28, 2020, which is hereby incorporated by reference in its entirety.

### STATEMENT REGARDING FEDERALLY SPONSORED RESEARCH OR DEVELOPMENT

This invention was made with government support under Grant No. DGE-1656466, awarded by the National Science Foundation.

### SEQUENCE LISTING

A Sequence Listing filed electronically herewith is also hereby incorporated by reference in its entirety (File Name: PRIN-76076_ST25.txt; Date Created: May 27, 2021; File Size: 14 kilobytes.)

### BACKGROUND

Living cells have evolved strategies for organizing their contents by compartmentalizing specific sets of biomolecules into a variety of different organelles. In addition to vesicle-like organelles, there are multiple types of intracellular bodies that are not membrane-bound, for example the nucleolus, stress granules, processing bodies and signaling clusters. These structures, referred to as membrane-less organelles or condensates, represent dynamic molecular assemblies, which can play various roles in living cells such as sequestering biomolecules, facilitating chemical reactions and channeling intracellular signaling.

### SUMMARY

Provided herein are intracellular protein condensates, designated “Corelets”, which function as organizational elements within biological systems. In some embodiments, sub-micron-scale protein clusters form without a delineating membrane, representing membrane-less assemblies which arise through liquid-liquid phase separation. In some embodiments, protein condensates form phase-separated clusters, e.g., in a cell. In some embodiments, condensate systems may be engineered to induce desirable cellular effect for systems of biotechnological applications. The programmable condensate system can enable modular targeting of specific proteins. In some embodiments, protein-peptide interaction domains (PPIDs) are incorporated to functionalize engineered condensates with the attributes of a recruited protein of interest, resulting in a modular system that allows for diverse facile and reprogrammable applications, including in enzyme clustering of metabolic pathways. Colocalizing specific metabolic enzymes in these condensates results in functionalized organelles with which can be used to manipulate the output of engineered metabolic pathways for the production of a pharmaceutical precursor.

Provided herein is a composition comprising: a first fusion protein comprising a target protein fused to a peptide ligand; and at least one additional fusion protein comprising either (a) a second fusion protein comprising a self-assembling protein and at least one protein-peptide interaction domain (PPID); or (b) a second fusion protein comprising a self-assembling protein, and a third fusion protein comprising a low complexity or intrinsically disordered protein region (IDR), wherein either the second fusion protein or the third fusion protein further comprises at least one PPID, wherein the peptide ligand is capable of binding to the at least one PPID.

In some embodiments, the composition is configured to form an assembled phase, the assembled phase comprising at least one aggregate. In some embodiments, the aggregate comprises phase-separated clusters.

In some embodiments, the peptide ligand binds to the PPID, thereby recruiting the target protein to the phase-separated clusters.

In some embodiments, the phase-separated clusters form upon exposure of the at least one additional fusion protein to a stimulus selected from the group consisting of light, temperature, chemicals, and any combination thereof.

In some embodiments, the composition is present in a cell. In some embodiments, the composition increases production of at least one chemical in the cell as compared with a cell that does not contain the composition.

In some embodiments, the target protein is an enzyme. In some embodiments, the enzyme is an enzyme of a metabolic pathway.

In some embodiments, the target protein is a fluorescent protein.

In some embodiments, the at least one additional fusion protein further comprises at least one fluorescent tag.

In some embodiments, the composition comprises a plurality of first fusion proteins.

In some embodiments, the at least one additional fusion protein comprises a second fusion protein comprising the self-assembling protein, and a third fusion protein comprising the low complexity or intrinsically disordered protein region (IDR).

In some embodiments, the second fusion protein comprises the self-assembling protein and a light-sensitive receptor protein; and the third fusion protein comprises the low complexity or intrinsically disordered protein region (IDR), and a cognate partner to the light-sensitive receptor protein. In some embodiments, the PPID is fused to the second fusion protein. In some embodiments, the PPID is fused to the third fusion protein.

In some embodiments, the light-sensitive receptor protein is iLID. In some embodiments, the cognate partner to the light-sensitive receptor protein is sspB. In some embodiments, the light-sensitive receptor protein is sensitive to at least one visible, ultraviolet (UV) or infrared (IR) wavelength of light. In some embodiments, the cognate partner of the light-sensitive receptor protein is configured to bind to the light-sensitive receptor protein when the system is irradiated with at least one wavelength of light.

In some embodiments, the second fusion protein and/or the third fusion protein further comprises a fluorescent tag.

In some embodiments, the second fusion protein comprises a first fluorescent tag fused to the light-sensitive receptor protein, the self-assembling protein and the PPID; and the third fusion protein comprises a second fluorescent tag fused to the low complexity or intrinsically disordered protein region (IDR) and the cognate partner to the light-sensitive receptor protein.

In some embodiments, the second fusion protein comprises a first fluorescent tag fused to the light-sensitive receptor protein and the self-assembling protein; and the third fusion protein comprises a second fluorescent tag fused to the low complexity or intrinsically disordered protein region (IDR), the cognate partner to the light-sensitive receptor protein, and the PPID.

In some embodiments, the second fusion protein comprises the self-assembling protein and the at least one PPID; and the third fusion protein comprises the low complexity or intrinsically disordered protein region (IDR) and the self-assembling protein. In some embodiments, the second fusion protein and/or the third fusion protein further comprises a fluorescent tag. In some embodiments, the second fusion protein comprises a first fluorescent tag fused to the self-assembling protein and the at least one PPID; and the third fusion protein comprises a second fluorescent tag fused to the low complexity or intrinsically disordered protein region (IDR) and the self-assembling protein.

In some embodiments, the at least one additional fusion protein comprises a second fusion protein, the second fusion protein comprising the PPID and the self-assembling protein; and wherein the target protein is a self-interacting protein (which forms, e.g., dimers trimers, tetramers, and the like). In some embodiments, the second fusion protein further comprises a fluorescent tag.

In some embodiments, the at least one additional fusion protein comprises a second fusion protein, the second fusion protein comprising the PPID, the self-assembling protein, and the low complexity or intrinsically disordered protein region (IDR). In some embodiments, the second fusion protein further comprises a fluorescent tag.

Provided is a composition comprising a first fusion protein and at least one additional fusion protein as described herein, for use in recruiting the target protein to the at least one additional fusion protein.

Provided is a composition comprising a first fusion protein and at least one additional fusion protein as described herein, for use in enhancing a biosynthetic reaction by increasing a local concentration of the target protein, wherein the target protein is one or more enzymes of a metabolic pathway.

Provided is a method for increasing production of at least one chemical in a cell, the method comprising the steps of expressing, in the cell, a composition comprising a first fusion protein and at least one additional fusion protein as described herein, under conditions sufficient to form an assembled phase.

Provided is a method for enhancing a biochemical (e.g., biosynthetic) reaction in a cell, the method comprising the steps of expressing, in the cell, a composition comprising a first fusion protein and at least one additional fusion protein as described herein, under conditions sufficient to form an assembled phase.

Provided is a method of treating a condition or disorder in a subject, the method comprising the step of expressing a composition as comprising a first fusion protein and at least one additional fusion protein as described herein, in a cell of the subject under conditions sufficient for the at least one additional fusion protein to form an assembled phase. In some embodiments, the condition or disorder is a condition or disorder of a metabolic, signaling, a transcription, a translation, or degradation pathway.

Provided herein is a cell expressing the composition comprising a first fusion protein and at least one additional fusion protein as described herein. In some embodiments, the cell is a human cell, an animal cell, or a yeast cell.

Provided herein is an engineered system comprising a plurality of nucleic acids encoding the fusion proteins as described herein.

Provided herein is an engineered system comprising a first nucleic acid construct encoding a target protein fused to a peptide ligand; and at least one additional nucleic acid construct comprising: (a) a second nucleic acid construct encoding a self-assembling protein and at least one protein-peptide interaction domain (PPID); or (b) a second nucleic acid construct encoding a self-assembling protein, and a third nucleic acid construct encoding a low complexity or intrinsically disordered protein region (IDR), wherein either the second nucleic acid construct or third nucleic acid construct further encode at least one PPID; wherein, when expressed in a cell, the peptide ligand is capable of binding to the at least one PPID.

In some embodiments, the first nucleic acid construct or the at least one additional nucleic acid construct further comprises a promoter.

In some embodiments, the first nucleic acid construct or the at least one additional nucleic acid construct further comprises a sequence encoding a polyadenylation tail.

In some embodiments, the first nucleic acid construct or the at least one additional nucleic acid construct further comprises an origin of replication.

In some embodiments, the first nucleic acid construct or the at least one additional nucleic acid construct comprises a sequence encoding a 5′ untranslated region (5′-UTR).

In some embodiments, the first nucleic acid construct or the at least one additional nucleic acid construct comprises a sequence encoding a 3′ untranslated region (3′-UTR).

In some embodiments, the first construct or the at least one additional construct further comprises a restriction site. In some embodiments, the restriction site is a NotI, XhoI, SpeI, EcoRI, BamHI, HinFI, XbaI, NheI, MreI, XmaI, AgeI, BspEI, PacI, PmeI, KpnI, SacI, or AscI restriction enzyme cutting site.

In some embodiments, the system according to the present disclosure is expressed in a cell, wherein at least one additional fusion protein is configured to form an assembled phase, the assembled phase comprising at least one aggregate. In some embodiments, the aggregate comprises phase-separated clusters.

In some embodiments, the system is expressed. In some embodiments, expression of the system increases production of at least one chemical in the cell as compared with a cell that does not contain the system.

In some embodiments, the target protein encoded by the first fusion protein is an enzyme. In some embodiments, the enzyme is an enzyme of a metabolic pathway.

In some embodiments, the target protein encoded by the first fusion protein is a fluorescent protein.

In some embodiments, the at least one additional construct further encodes at least one fluorescent tag.

In some embodiments, the system comprises a plurality of first constructs.

In some embodiments, the at least one additional nucleic acid construct comprises a second nucleic acid construct encoding the self-assembling protein, and a third nucleic acid construct encoding the low complexity or intrinsically disordered protein region (IDR).

In some embodiments, the second nucleic acid construct encodes the self-assembling protein and a light-sensitive receptor protein; and the third nucleic acid construct encodes the low complexity or intrinsically disordered protein region (IDR), and a cognate partner to the light-sensitive receptor protein. In some embodiments, the second nucleic acid construct encodes the PPID. In some embodiments, the third nucleic acid construct encodes the PPID.

In some embodiments, the light-sensitive receptor protein is iLID. In some embodiments, the cognate partner to the light-sensitive receptor protein is sspB. In some embodiments, the light-sensitive receptor protein is sensitive to at least one visible, ultraviolet (UV) or infrared (IR) wavelength of light.

In some embodiments, the system is expressed in a cell, and the cognate partner of the light-sensitive receptor protein is configured to bind to the light-sensitive receptor protein when the system is irradiated with at least one wavelength of light.

In some embodiments, the second nucleic acid construct and/or the third nucleic acid construct further encodes a fluorescent tag.

In some embodiments, the second nucleic acid construct encodes a first fluorescent tag fused to the light-sensitive receptor protein, the self-assembling protein and the PPID; and the third nucleic acid construct encodes a second fluorescent tag fused to the low complexity or intrinsically disordered protein region (IDR) and the cognate partner to the light-sensitive receptor protein.

In some embodiments, the second nucleic acid construct encodes a first fluorescent tag fused to the light-sensitive receptor protein and the self-assembling protein; and the third nucleic acid construct encodes a second fluorescent tag fused to the low complexity or intrinsically disordered protein region (IDR), the cognate partner to the light-sensitive receptor protein, and the PPID.

In some embodiments, the second nucleic acid construct encodes a self-assembling protein and at least one PPID; and the third nucleic acid construct encodes a low complexity or intrinsically disordered protein region (IDR), and a self-assembling protein.

In some embodiments, the second nucleic acid construct and/or the third nucleic acid construct further encodes a fluorescent tag.

In some embodiments, the second nucleic acid construct encodes a first fluorescent tag fused to the self-assembling protein and the at least one PPID; and the third nucleic acid construct encodes a second fluorescent tag fused to the low complexity or intrinsically disordered protein region (IDR) and the self-assembling protein.

In some embodiments, the at least one additional nucleic acid construct comprises a second nucleic acid construct, the second nucleic acid construct encoding the PPID and the self-assembling protein; and wherein the target protein is a self-interacting protein. In some embodiments, the second nucleic acid construct further encodes a fluorescent tag.

In some embodiments, the at least one additional nucleic acid construct comprises a second nucleic acid construct, the second fusion construct encoding the PPID, the self-assembling protein, and the low complexity or intrinsically disordered protein region (IDR). In some embodiments, the second nucleic acid construct further encodes a fluorescent tag.

Provided is a system a first nucleic acid construct and at least one additional nucleic acid construct as described herein, for use in recruiting the target protein to the at least one additional fusion protein.

Provided is a system a first nucleic acid construct and at least one additional nucleic acid construct as described herein, for use in enhancing a biosynthetic reaction by increasing a local concentration of the target protein, wherein the target protein is one or more enzymes of a metabolic pathway.

In some embodiments relating to the compositions and/or engineered systems described herein, the fluorescent protein tag is m-Cherry, Green Fluorescent Protein (GFP), enhanced GFP (EGFP), Cyan Fluorescent Protein (CFP), Yellow Fluorescent Protein (YFP), Red Fluorescent Protein (RFP), Orange Fluorescent Protein (OFP), blue fluorescent protein (BFP), tetracysteine fluorescent motif, or any combination thereof.

In some embodiments relating to the compositions and/or engineered systems described herein, the PPID is selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

In some embodiments relating to the compositions and/or engineered systems described herein, the peptide ligand is a peptide capable of binding to a PPID selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

In some embodiments relating to the compositions and/or engineered systems described herein, the intrinsically disordered protein region (IDR) is FUS or FUSn.

In some embodiments relating to the compositions and/or engineered systems described herein, the self-assembling protein is ferritin. In some embodiments, the ferritin is a ferritin heavy chain or a ferritin light chain. In some embodiments, the ferritin is a ferritin heavy chain.

Provided herein is a method of forming an assembled phase, the method comprising expressing, within a cell, a composition comprising a first fusion protein and at least one additional fusion protein as described herein, and allowing the composition to undergo phase separation into at least one assembled (e.g., condensed) phase within the living cell. In some embodiments, the at least one assembled (e.g., condensed) phase comprises phase-separated clusters.

Provided herein is a method for screening for protein-protein interactions, the method comprising expressing, in a cell, a first composition comprising a first target protein as described herein, and a second composition comprising a second target protein as described herein, under conditions sufficient to form an assembled phase; and measuring colocalization of the first and the second target proteins in the cell.

Provided herein is a method of screening for compounds capable of modulating a protein-protein interaction, the method comprising: expressing, in a cell, a first composition comprising a first target protein as described herein, and a second composition comprising a second target protein as described herein, under conditions sufficient to form an assembled phase; contacting the cell with a test compound; measuring colocalization of the first and the second target proteins in the cell in the presence of the test compound; and comparing colocalization of the first and the second target proteins in the presence of the test compound to a reference sample measured in the absence of the test compound; wherein a change in the colocalization of the first and the second target proteins in the presence of the test compound as compared with the reference sample is indicative of the ability of the test compound to modulate said protein-protein interaction.

In some embodiments, measuring colocalization of the first and the second target proteins comprises detecting location of the first and the second target proteins in the cell. In some embodiments, detecting comprises imaging. In some embodiments, detecting location of the first and the second target proteins in the cell comprises detecting a signal. In some embodiments, the signal is an electronic signal or an electromagnetic signal. In some embodiments, the signal is optically detectable. In some embodiments, the optically detectable signal is a fluorescence signal or a luminescence signal. In some embodiments, the optically detectable signal is a small-molecule dye, a fluorescent molecule or protein, a quantum dot, a colorimetric reagent, a chromogenic molecule or protein, a Raman label, a chromophore, or a combination thereof.

In some embodiments, measuring colocalization of the first and the second target proteins comprises determining presence or amount of a compound in a biological pathway.

Provided herein is a method of screening for protein-nucleic acid interactions, the method comprising: expressing, in a cell, a composition comprising a target protein as describe herein, under conditions sufficient to form an assembled phase; and measuring binding of a nucleic acid to the target protein in the cell.

Provided herein is a method of screening for compounds capable of modulating a protein-nucleic acid interaction, the method comprising: expressing, in a cell, a composition a target protein as described herein, under conditions sufficient to form an assembled phase; contacting the cell with a test compound; measuring binding of a nucleic acid to the target protein in the cell in the presence of the test compound; and comparing binding of the nucleic acid to the target protein in the presence of the test compound to a reference sample measured in the absence of the test compound; wherein a change in the binding of the nucleic acid to the target protein in the presence of the test compound as compared with the reference sample is indicative of the ability of the test compound to modulate protein-nucleic acid interaction.

In some embodiments, measuring binding of the nucleic acid to the target protein comprises detecting a signal from the nucleic acid. In some embodiments, the measuring further comprises, prior to detecting, staining the nucleic acid or binding the nucleic acid with a detectable probe. In some embodiments, the signal is a fluorescence signal or a luminescence signal.

In some embodiments, measuring binding of the nucleic acid to the target protein comprises determining presence or amount of a compound in a biological pathway.

In some embodiments, the compound disrupts binding of the target protein to the nucleic acid. In other embodiments, the compound enhances binding of the target protein to the nucleic acid.

Provided herein is a method of screening for a compound capable of modulating a target protein, the method comprising expressing, in a cell, a composition comprising a first fusion protein and at least one additional fusion protein as described herein, under conditions sufficient to form an assembled phase; contacting the cell with a test compound; measuring a biological parameter of the target protein; and comparing said biological parameter to a reference sample measured in the absence of the test compound; wherein a change in the biological parameter in the presence of the test compound as compared with the reference sample is indicative of the ability of the test compound to modulate said target protein. In some embodiments, the biological parameter is enzymatic activity, metabolism, signaling, transcription, translation, degradation, a post-translational modification, or presence or amount of a compound in a biological pathway.

In some embodiments, modulating the target protein comprises inhibiting the amount of activity of the target protein. In some embodiments, modulating the target protein comprises activating or increasing the amount or activity of the target protein.

In some embodiments, the method further comprises administering to a subject the test compound to treat a condition.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The present disclosure explicitly incorporates by reference U.S. Pat. No. 10,538,756 and U.S. patent application Ser. No. 16/704,115.

### BRIEF DESCRIPTION OF DRAWINGS

FIG. 1 illustrates modular platform for spatiotemporal control of proteins via PPIDs. FIG. 1A, 1B: Recruitment to light-inducible Corelet system. FIG. 1C: Recruitment to constitutive Corelet system. FIG. 1D: C-terminus and N-terminus peptide (PEP)-tagged protein. FIG. 1E: The constitutive Corelet system can be expressed as a mixed population of self-assembling proteins fused with low complexity or intrinsically disordered protein regions (IDRs) and PPIDs. FIG. 1F: When the recruited target protein is self-interacting (e.g., forms a dimer, trimer, a tetramer or an oligomer), the IDR can be omitted from the Corelet design, as shown herein for constitutive Corelets, and still yield cluster formation enriched in tagged cargo.

FIG. 2 illustrates a modular platform for spatiotemporal control of enzymes via PPIDs. FIG. 2A, 2B: Recruitment to light-inducible Corelet system. Light-induction is based on an increase in iLID-sspB affinity in blue light. Twenty-four human ferritin domains (hFTH1) self-assemble to spherical cores that recruit IDRs upon blue-light activation. PPIDs can be incorporated into the FTH1 (Core) or IDR construct, allowing for cargo recruitment. Multimerized IDR-based self-interactions lead to the formations of larger clusters, shown in (C), capable of recruiting cargo with the PPIDs. FP: fluorescent protein tag. FIG. 2C: Recruitment to a constitutive variant of the Corelet system. FIG. 2D: Cargo can be co-expressed with N-terminal or C-terminal peptide tags, specific to the PPIDs used in the Corelets to lead to recruitment to clusters. FIG. 2E: The constitutive Corelet system can be expressed as a mixed population of self-assembling protein (e.g., ferritins) fused with IDRs and PPIDs. FIG. 2F: When the recruited cargo is self-interacting (e.g., forms a dimer, trimer, tetramer, or oligomer), the IDR can be omitted from the Corelet design, as shown here for constitutive Corelets, and still yield cluster formation enriched in tagged cargo. All schematics created with Biorender. FIG. 2G: Time series of yeast cells expressing light-induced Corelets. IDR used is from the human FUS protein, N-terminus (hFUSn). Light on from 0-6 minutes (underlined); Cell outline, membrane dye. FIG. 2H: hFUSn Constitutive Corelets, maximum Z-projection. Cell outline, membrane dye. FIG. 2I: Constitutive Corelets without IDRs, but recruiting dimeric proteins with PDZ PPID, maximum Z-projection. Cell outline, membrane dye. All scale bar, 5 μm.

FIG. 3. GFP cargo recruitment to mCherry (mCh)-tagged constitutive Corelet (FC) fused to the PPID listed on the left. GFP is tagged with that domain's corresponding peptide as a C-terminal (left) or N-terminal (right) fusion. Scale bar, 5 μm.

FIG. 4. PPIDs show orthogonal enrichment of proteins tagged with corresponding peptides in Light Induced Corelets. Data shown as a heat map of the resulting mean Pearson correlation coefficient for each PPID-peptide pair in this interaction matrix. Peptide tag used shown on the left, and domain in the clustering construct labelled at the bottom.

FIG. 5. PPIDs show orthogonal enrichment of proteins tagged with corresponding peptides in Constitutive Corelets. Data shown as a heat map of the resulting mean Pearson correlation coefficient for each PPID-peptide pair in this interaction matrix. Peptide tag used shown on the left, and domain in the clustering construct labelled at the bottom.

FIG. 6. Enzyme enrichment with PPIDs in constitutive Corelets increase shikimate production. FIG. 6A: ARO4 condenses PEP, produced by ENO2, and E4P produced by TKL1 and TAL1, to produce DAHP, which is converted to Shikimate, a notable precursor to natural aromatic amino acids and derivatives of them (AAA). FIG. 6B: TAL1 and ARO4 recruited to clusters with PDZ tag and domain; TKL1 and ENO2 recruited by PTB tag and domain. When all four enzymes are recruited to the cluster through expression with PTB/PDZ tags (+), production is enhanced compared to when certain enzymes are removed from the cluster through expression without fusion to PTB/PDZ tag (−).

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. Unless otherwise clear from context, all numerical values provided herein are modified by the term about.

Ranges provided herein are meant to include all of the values within the range. For example, a range of 1 to 10 is understood to include any number, combination of numbers, or sub-range from the group consisting of 1, 2, 3, 4, 5, 6, 7, 8, 9, or 10, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either endpoint of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless otherwise defined, all technical and scientific terms used herein have the same meaning as commonly understood by one of ordinary skill in the art to which this disclosure belongs. Although methods and materials similar or equivalent to those described herein can be used in the practice or testing various aspects described herein, suitable methods and materials are described below.

### Definitions

The term “nucleic acid” as used herein generally refers to one or more nucleobases, nucleosides, or nucleotides, and the term includes polynucleobases, polynucleosides, and polynucleotides.

The term “polynucleotide”, as used herein generally refers to a molecule comprising two or more linked nucleic acid subunits, e.g., nucleotides, and can be used interchangeably with “oligonucleotide”. For example, a polynucleotide may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides include nucleotides in which the sugar is ribose. Deoxyribonucleotides include nucleotides in which the sugar is deoxyribose. A nucleotide can be a nucleoside monophosphate, nucleoside diphosphate, nucleoside triphosphate or a nucleoside polyphosphate. For example, a nucleotide can be a deoxyribonucleoside polyphosphate, such as a deoxyribonucleoside triphosphate (dNTP), Exemplary dNTPs include deoxyadenosine triphosphate (dATP), deoxycytidine triphosphate (dCTP), deoxyguanosine triphosphate (dGTP), uridine triphosphate (dUTP) and deoxythymidine triphosphate (dTTP). dNTPs can also include detectable tags, such as luminescent tags or markers (e.g., fluorophores). For example, a nucleotide can be a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. Exemplary polynucleotides include, but are not limited to, short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), and heteronuclear RNA (hnRNA), and encompasses both the nucleotide sequence and any structural embodiments thereof, such as single-stranded, double-stranded, triple-stranded, helical, hairpin, stem loop, bulge, etc. In some cases, a polynucleotide is circular. A polynucleotide can have various lengths. For example, a polynucleotide can have a length of at least about 7 bases, 8 bases, 9 bases, 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. For example, polynucleotide sequences may comprise isolated and purified DNA/RNA molecules, synthetic DNA/RNA molecules, and/or synthetic DNA/RNA analogs.

The terms “polypeptide”, “protein” and “peptide” as used herein interchangeably, refer to a polymer of amino acid residues linked via peptide bonds and which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant.

Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

A “self-assembling protein” or “SAP” means a protein capable of forming an organized multimer without participation of external mediators. Self-assembling proteins are capable of take a defined physical arrangement without external stimuli. In some embodiments, self-assembly proteins may be intramolecular self-assembling proteins. In other embodiments, self-assembling proteins may be intermolecular self-assembly proteins.

A “self-interacting protein” or “SIP” means a protein which, through inter-protein interactions, is capable of forming aggregates such as multimers, e.g., dimers, trimers, tetramers, or oligomeric structures. The formed multimers may allow for larger, mesoscale, cluster assembly or puncta formation and/or phase separation. Self-interacting proteins may comprise structurally defined domains, such as proteins that form dimers, trimers, tetramers, or oligomers, or structurally undefined such as the IDRs.

An “assembled phase” means formation of a separate phase, e.g., in a cell, such as, without limitation, formation of liquid-liquid phase-separated clusters.

A “phase separated cluster” refers to an agglomerate colonized by the interaction between particles formed by self-assembly of the self-assembled proteins.

A “light-inducible protein” refers to a protein forming a heterodimer with other proteins when irradiated with light having a particular wavelength.

A “cognate-partner of a light-inducible protein” refers to a target protein forming a heterodimer with the light-induced heterodimerized protein” when irradiated with light having a particular wavelength.

A “fusion protein” refers to a protein in which two or more proteins are connected each other by amino bond while maintaining the functionality of each unit protein.

A “heterodimer” refers to a dimer or a complex formed by two different proteins via the interaction between them.

A “homodimer refers to a dimer or a complex formed by two same proteins via intermolecular interaction.

The term “operably linked to”, as used herein, means that a particular polynucleotide can function when connected to other polynucleotides. In other words, “a polynucleotide encoding a particular protein, wherein the polynucleotide is operably linked to the promoter” means that the polynucleotide can be transcribed into mRNAs according to the action of the promoter and the mRNAs are translated to the protein. Thus, the term “a polynucleotide encoding a particular protein is operably linked to a polynucleotide encoding the other protein” means that the particular protein can be expressed as fused to the other protein.

The term “construct,” as used herein, refers to a recombinant nucleic acid that has been generated for the purpose of the expression of a specific nucleotide sequence(s), or is to be used in the construction of other recombinant nucleotide sequences. The construct can be a vector or a plasmid. The construct can be a recombinant nucleic acid molecule that can be propagated and used to transfer nucleic acid segment(s) from one organism to another. Vectors can comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors can be recombinant nucleic acid molecules, often derived from bacteriophages, or plant or animal viruses. Plasmids and cosmids can refer to two such recombinant vectors. A nucleic acid vector can be a linear molecule, or in circular form, depending on type of vector or type of application. Some circular nucleic acid vectors can be linearized prior to delivery into a cell (e.g., a host cell).

The terms “enhance” or “enhancing,” as used herein, means to increase or prolong either in amount, potency or duration a desired effect. For example, in regard to enhancing concentration of a protein the term “enhancing” can refer to the ability to increase local cellular concentration either in amount, potency or duration, of a the protein, e.g., an enzyme.

### PPIDs Recruit Peptide-Tagged Cargo to Engineered Corelets

The present disclosure describes systems and methods for nucleation of phase separation in biological samples, to generate a clustering system as a means to recruit target proteins (e.g., enzymes) to resulting aggregates, e.g., phase-separated condensates. The presently disclosed platform utilizes a combination of (a) an independent method of forming highly concentrated proteinaceous clusters in a biological sample; and (b) a technology to recruit target proteins of interest (e.g., enzymes) to the clusters without direct fusion to the constructs forming the condensates.

In some embodiments, the presently disclosed platform consists, consists essentially of, or comprises a library of nucleic acid constructs (e.g., DNA constructs or RNA constructs) and the biological organisms they are introduced into to allow for expression of proteins encoded by the constructs (e.g., DNA or RNA constructs). The constructs can comprise two components. The first component can encode condensate-forming protein units fused to one or more protein-peptide interaction domains (PPIDs) that are able to recruit proteins tagged with a corresponding peptide(s) recognized by the PPIDs. The condensate-forming protein units may be additionally fused to, or replaced with, other protein domains that make condensate formation inducible by specific stimuli (e.g. light, chemicals, temperature, etc.). The second component can encode peptide(s) capable of being recognized by the PPIDs, fused to a protein of interest, e.g., an enzyme. The protein-peptide fusion protein encoded by the constructs can allow for facile and modular tagging of a protein of interest with peptides that interact and recruit to the engineered condensates.

In some embodiments, the presently disclosed platform consists, consists essentially of, or comprises compositions comprising a library of engineered fusion proteins encoded by the constructs (e.g., DNA or RNA constructs) disclosed herein. The composition can comprise two components. The first component can comprise condensate-forming protein units fused to one or more PPIDs that are able to recruit proteins (e.g., enzymes or other target proteins of interest) tagged with a corresponding peptide(s) recognized by the PPIDs. The second component can comprise peptide(s) capable of being recognized by the PPIDs, fused to a protein of interest, e.g., an enzyme. The protein-peptide fusion protein can allow for facile tagging of a protein of interest with peptides that interact and recruit to the engineered condensates.

In some embodiments, PPIDs are incorporated into recombinant proteins that specifically interact with small peptide sequences that can be largely incorporated onto termini of proteins, with minimal effect on protein activity, to allow the engineered condensate to be widely functionalized with the recruited protein(s) activity.

The disclosed platform can be advantageous compared with other approaches in that it represents a dynamically controlled, “plug-and-play” (i.e. readily reconfigurable) system for concentrating specific proteins including enzymes and tailoring their localization, to manipulate intracellular interactions and biochemistry.

The plug-and-play nature of the proposed system has broad potential utilizations. The system may be used to study the interaction of a particular subset of enriched proteins in puncta. This could be notably relevant in the study and engineering of signaling pathways, some of which are thought to be modulated via biomolecular condensates. In some embodiments, the platforms may be used to temporarily sequester proteins in these condensates to reduce the effects of their activity in the rest of the cytosol or other subcellular compartments.

In some embodiments, the disclosed platforms can be used for enrichment of enzymes of a metabolic pathway to enhance metabolic output of the pathway. This occurs, e.g., through minimization of intermediate loss to competing pathways and promoting the desired directionality of reversible reactions. In some embodiments, different enzymes of a particular pathway may be recruited simultaneously or sequentially to condensates, to enhance local concentration of such enzymes and thereby facilitate metabolic output. Application of the platform to a natural product biosynthesis pathway as disclosed herein exemplifies the ability of this approach to minimize the loss of intermediates to competing pathways. It is apparent to those skilled in the art that the utility of the disclosed platform is not limited to biosynthetic applications, and that the platform may be used in a wide range of biotechnology applications including facilitating therapeutic discovery within human and non-human cell systems.

In some embodiments, the disclosed platform can be used to modulate chemical biosynthesis, signaling, metabolism, transcription, translation, or degradation, in any number of different diagnostic, therapeutic, or screening applications. In some embodiments, the disclosed platform can be used to screen for protein-protein interactions. In some embodiments, the disclosed platform can be used screen for compounds capable of modulating a protein. In some embodiments, the disclosed platform can be used to treat a condition or disorder that is associated with a target protein.

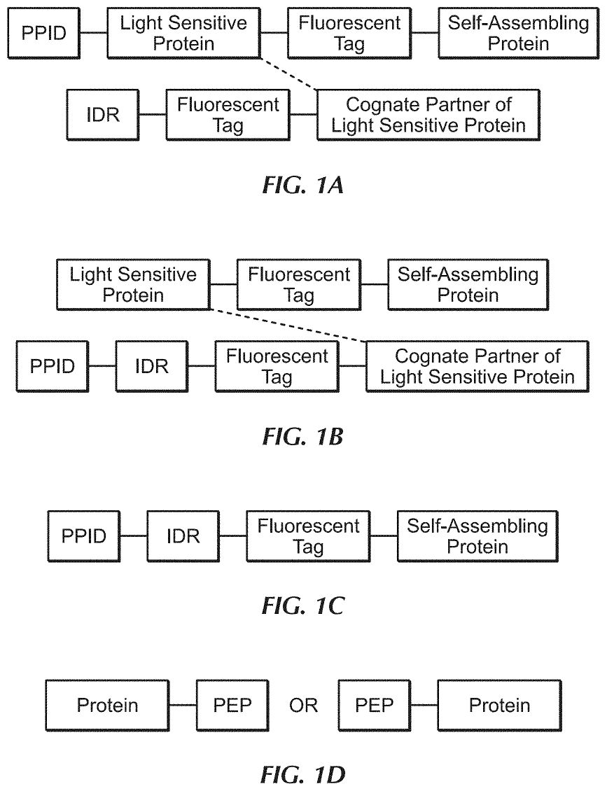

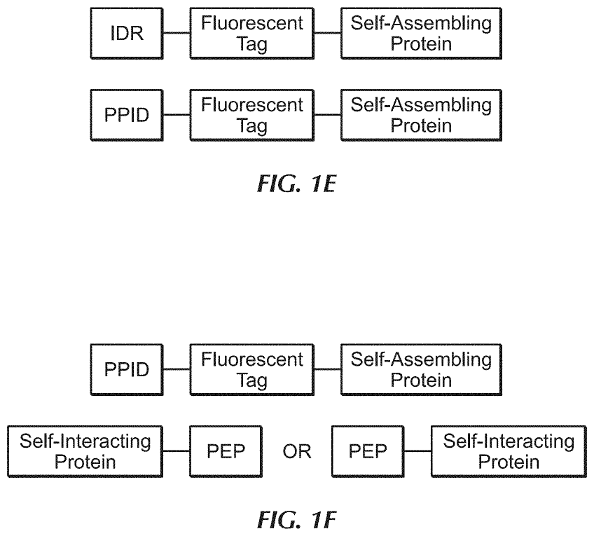

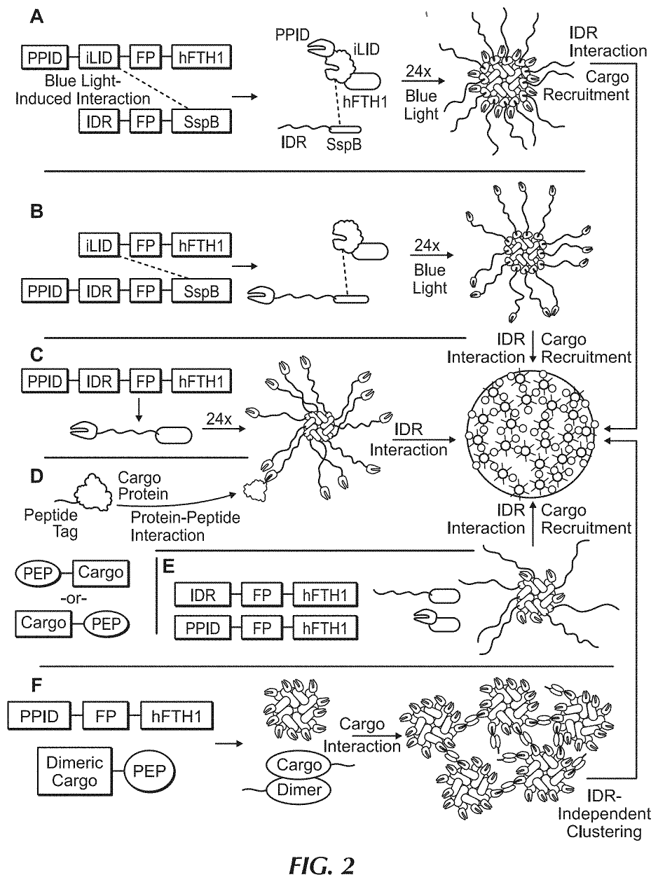

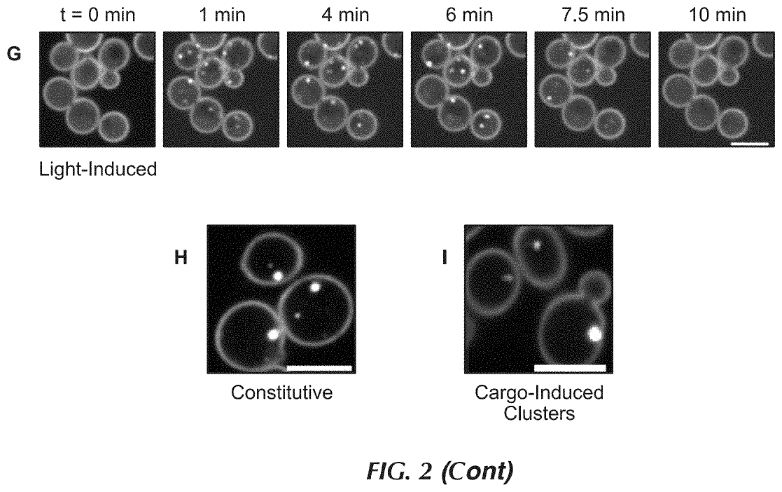

Multiple PPIDs, identified for their ability to interact with small peptides outside of their original biological context, may be used in both light-induced Corelets (FIG. 1A-B, 2A-B), or constitutive Corelets (FIG. 1C, 1E, 1F, 2C, 2E, 2F) expressed, e.g., in yeast, e.g., Saccharomyces cerevisiae. These PPIDs may be incorporated into clustering systems in multiple systems, as shown exemplarily in FIG. 1 and FIG. 2. As demonstrated herein, each system shown demonstrates the ability to recruit proteins tagged with the corresponding peptide into the clusters they form.

FIG. 1A-B depict a general embodiment of a light-sensitive Corelet system. The system generally comprises two fusion proteins and/or the corresponding nucleic acid constructs encoding the fusion proteins. One fusion protein (FIG. 1A-B, top) comprises at least one light-sensitive receptor protein fused to a self-assembling protein subunit, which may be an oligomeric protein subunit. Another fusion protein (FIG. 1A-B, bottom) comprises at least one cognate partner of the light-sensitive receptor protein, fused to a full length or truncated low complexity or intrinsically disordered protein region (IDR). One of the fusion proteins further comprises a protein-peptide interaction domain (PPID). In some embodiments, the PPID is fused to the protein comprising the light-sensitive receptor protein, as shown in FIG. 1A. The PPID may be fused to the light-sensitive receptor protein according to the configuration shown in FIG. 1A, or in other locations as desired. In other embodiments, the PPID is fused to the protein comprising the cognate partner of the light-sensitive receptor protein, as shown in FIG. 1B. The PPID may be fused to the low complexity or intrinsically disordered protein region (IDR) as shown in FIG. 1B, or in other locations if desired. Upon irradiation of light, the light-sensitive receptor protein is configured to bind to its cognate partner to form an assembled state, e.g., an aggregate, a condensate, or phase-separated clusters. The system further comprises a fusion protein comprising a target protein fused to a peptide ligand (FIG. 1D). The peptide ligand may be fused to the C-terminus of the protein. Alternatively, the peptide ligand may be fused to the N-terminus of the protein. The peptide ligand is designed to recognize and bind to the PPID in the Corelet system, thereby recruiting the target protein to the Corelets.

Optionally, the Corelet may include a fluorescent protein tag (FP), as exemplarily indicated in FIG. 1A, 1B. For example, the fluorescent protein tag may be fused between the light-sensitive receptor protein and the self-assembling protein, or in other locations if desired. Alternatively or additionally, the fluorescent tag may be fused between the cognate partner of the light-sensitive receptor protein and the intrinsically disorderd protein, or in other locations if desired.

In other embodiments, in cases where photo-sensitivity is not desired, rather than using two proteins to create a photo-activatable or photo-deactivatable systems, a single, or multiple constitutive fusion protein(s) may be utilized. FIG. 1C depicts a general embodiment of a constitutive Corelet system. As shown in FIG. 1C, a single fusion protein may comprise a self-assembling protein subunit and a full length or truncated low complexity or intrinsically disordered protein region. In this manner, the system may generate a disordered protein-based seed for molecular clustering without requiring photo-activation or deactivation. The fusion protein further comprises a PPID, which is designed to recognize and bind to a peptide ligand as described above. In some embodiments, the PPID is fused to the intrinsically disordered protein region as shown in FIG. 1C. However, the PPID may be fused elsewhere in the fusion protein as desired. Optionally, the fusion protein may further include a fluorescent tag (FP), as exemplarily indicated in FIG. 1A, 1B. If included, the fluorescent tag may either be fused as indicated in FIG. 1C, i.e., between the IDR and the self-assembling protein, or in other locations if desired. The Corelet system is configured to form an assembled state and recruit a target protein (FIG. 1D) through interactions of the peptide ligand and PPID, as described above. In some embodiments, the disclosure provides nucleic acid constructs encoding the Corelet system represented in FIG. 1C.

In other embodiments, a constitutive Corelet system can be expressed as a mixed population of self-assembled proteins fused with IDRs and PPIDs, as shown in FIG. 1E. The system generally comprises a first fusion protein population comprising a self-assembling protein fused to a low complexity or intrinsically disordered protein, and a second fusion protein population comprising a self-assembling protein fused to a PPID. Optionally, the fusion protein may further include a fluorescent tag (FP), as exemplarily shown in FIG. 1E. If included, the fluorescent tag may either be fused as indicated in FIG. 1E, i.e., between the IDR and the self-assembling protein, or between the PPID and the self-assembling protein, or both, or in other locations if desired. The Corelet system is configured to form an assembled state and recruit a target protein (FIG. 1D) through interactions of the peptide ligand and PPID, as described above. In some embodiments, the disclosure provides nucleic acid constructs encoding the constitutive Corelet system represented in FIG. 1E.

In other embodiments, a constitutive Corelet system can be expressed without a low complexity or intrinsically disordered protein region (IDR), as shown exemplarily in FIG. 1F. For example, when the recruited cargo is self-interacting (e.g., forms a dimer, trimer, tetramer, and the like), the IDR can be omitted from the Corelet design, as shown herein for constitutive Corelets, and still yield cluster formation enriched in tagged cargo comprising a target protein. Optionally, the fusion protein may further include a fluorescent tag (FP), as exemplarily shown in FIG. 1F. If included, the fluorescent tag may either be fused as indicated in FIG. 1F, i.e., between the PPID and the self-assembling protein, or in other locations if desired. In accordance with this embodiment, the target protein (FIG. 1D) is self-interacting (e.g., forms dimers, trimers, tetramers or oligomers), and is recruited through interactions of the peptide ligand and PPID, as described above. In some embodiments, the disclosure provides nucleic acid constructs encoding the constitutive Corelet system represented in FIG. 1F.

FIG. 2 illustrates modular platform for spatiotemporal control of enzymes via PPIDs according to some embodiments of the present disclosure. FIG. 2A and 2B depict recruitment to light-inducible Corelet system. In some embodiments, Corelets comprise two modules: first, a self-assembling protein (e.g., ferritin heavy chain (hFTH1)) core functionalized by photo-activatable iLID domains (light-sensitive receptor protein), which is optionally tagged with a fluorescent protein (FP). Second, the Corelets comprise iLID's cognate partner, SspB, optionally fluorescent-tagged and conjugated to a self-interacting protein domain, such as the N-terminal IDR of FUS. Dashed line designate photo-inducible heterodimerizing units. In one embodiment depicted in FIG. 2A, the PPID is fused to the iLID as shown, or to any other component of the fusion protein. In another embodiment depicted in FIG. 2B, the PPID is fused to the fusion protein comprising the cognate partner of the light-sensitive receptor protein, SspB, e.g., to the IDR as shown, or to any other component of the fusion protein. In the active state, the light-sensitive receptor protein binds to the cognate partner of the light-sensitive receptor protein. In this example, the buried ssrA peptides become uncaged. Exposed ssrA rapidly bind their cognate sspB partners. Because the cognate partners are bound to an LCS/IDR, the clustering of LCS/IDR around the self-assembled core leads to the formation of a photo-stabilized liquid droplet as shown in FIG. 2A-B. The phase-stabilized liquid droplet may continue to grow to a larger phase-stabilized liquid droplet by recruiting single molecules, such as additional second proteins, or endogenous LCS/IDRs or other proteins not fused to a cognate partner. The phase-stabilized liquid droplet may also continue to grow via addition of single proteins, single core particles, or coalescence of mature multi-core particles. If included, the optional fluorescent tag may either be fused as indicated in 2A-B or in other locations if desired.

FIG. 2C, 2E, 2F depict recruitment to constitutive Corelet systems. In some embodiments, Corelets comprise a self-assembling protein (e.g. ferritin heavy chain) conjugated to a self-interacting protein domain, such as the N-terminal IDR of FUS (FIG. 2C). A PPID is fused to the IDR as shown, or to any other component of the fusion protein. The phase-stabilized liquid droplet may continue to grow to a larger phase-stabilized liquid droplet, as shown. In other embodiments, the constitutive Corelet system can be expressed as a mixed population of ferritins fused with IDRs and PPIDs (FIG. 2E). In other embodiments, when the recruited cargo is self-interacting (e.g., forms a dimer, trimer, tetramer and the like), the IDR can be omitted from the Corelet design, as shown here for constitutive Corelets, and still yield cluster formation enriched in tagged cargo (FIG. 2F). A fluorescent protein tag (FP) may optionally be included. If included, the optional fluorescent tag may either be fused as indicated in 2C, E, F or in other locations if desired.

FIG. 2D shows that target proteins (cargo) can be co-expressed with N-terminal or C-terminal peptide tags, specific to the PPIDs used in the Corelets to lead to recruitment to clusters. The Corelet systems depicted, e.g., in FIG. 2A, B, C, E, F, are configured to form phase separated droplets and recruit the cargo proteins through interactions of the peptide tag with the PPID.

### Embodiments of Engineered Corelets

Provided herein is a composition comprising: a first fusion protein comprising a target protein fused to a peptide ligand; and at least one additional fusion protein comprising either (a) a second fusion protein comprising a self-assembling protein and at least one protein-peptide interaction domain (PPID); or (b) a second fusion protein comprising a self-assembling protein, and a third fusion protein comprising a low complexity or intrinsically disordered protein region (IDR), wherein either the second fusion protein or the third fusion protein further comprises at least one PPID, wherein the peptide ligand is capable of binding to the at least one PPID. In some embodiments, the composition is configured to form an assembled phase, the assembled phase comprising at least one aggregate. In some embodiments, the aggregate comprises phase-separated clusters. In some embodiments, the peptide ligand binds to the PPID, thereby recruiting the target protein to the phase-separated clusters. In some embodiments, the peptide ligand binds to the PPID, thereby recruiting the target protein to the phase-separated clusters. In some embodiments, the at least one additional fusion protein further comprises at least one fluorescent tag. In some embodiments, the composition comprises a plurality of first fusion proteins.

Provided herein is a composition comprising: a first fusion protein comprising a target protein fused to a peptide ligand; a second fusion protein comprising a self-assembling protein, and a third fusion protein comprising a low complexity or intrinsically disordered protein region (IDR), wherein either the second fusion protein or the third fusion protein further comprises at least one PPID, and wherein the peptide ligand is capable of binding to the at least one PPID.

Provided herein is a composition comprising: a first fusion protein comprising a target protein fused to a peptide ligand; a second fusion protein comprising a self-assembling protein and a light-sensitive receptor protein; and a third fusion protein comprising ae low complexity or intrinsically disordered protein region (IDR), and a cognate partner to the light-sensitive receptor protein. In some embodiments, the PPID is fused to the second fusion protein. In some embodiments, the PPID is fused to the third fusion protein.

In some embodiments, the light-sensitive receptor protein is iLID. In some embodiments, the cognate partner to the light-sensitive receptor protein is sspB. In some embodiments, the light-sensitive receptor protein is sensitive to at least one visible, ultraviolet (UV) or infrared (IR) wavelength of light. In some embodiments, the cognate partner of the light-sensitive receptor protein is configured to bind to the light-sensitive receptor protein when the system is irradiated with at least one wavelength of light.

In some embodiments, the second fusion protein and/or the third fusion protein further comprises a fluorescent tag. In some embodiments, the second fusion protein comprises a first fluorescent tag fused to the light-sensitive receptor protein, the self-assembling protein and the PPID; and the third fusion protein comprises a second fluorescent tag fused to the low complexity or intrinsically disordered protein region (IDR) and the cognate partner to the light-sensitive receptor protein. In some embodiments, the second fusion protein comprises a first fluorescent tag fused to the light-sensitive receptor protein and the self-assembling protein; and the third fusion protein comprises a second fluorescent tag fused to the low complexity or intrinsically disordered protein region (IDR), the cognate partner to the light-sensitive receptor protein, and the PPID.

Provided herein is a composition comprising: a first fusion protein comprising a target protein fused to a peptide ligand, a second fusion protein comprising a self-assembling protein and at least one protein-peptide interaction domain (PPID); and a third fusion protein comprising a self-assembling protein and a low complexity or intrinsically disordered protein region (IDR), wherein the peptide ligand is capable of binding to the at least one PPID. In some embodiments, the second fusion protein and/or the third fusion protein further comprises a fluorescent tag. In some embodiments, the second fusion protein comprises a first fluorescent tag fused to the self-assembling protein and the at least one PPID; and the third fusion protein comprises a second fluorescent tag fused to the low complexity or intrinsically disordered protein region (IDR) and the self-assembling protein.

Provided herein is a composition comprising: a first fusion protein comprising a target protein fused to a peptide ligand, and a second fusion protein comprising a protein-peptide interaction domain (PPID) and a self-assembling protein, wherein the peptide ligand is capable of binding to the at least one PPID. In accordance with this embodiment, the target protein is preferably a self-interacting protein. In some embodiments, a self-interacting protein can form dimers. In some embodiments, a self-interacting protein can form trimers. In some embodiments, a self-interacting protein can form tetramers. In some embodiments, the second fusion protein further comprises a fluorescent tag.

Provided herein is a composition comprising: a first fusion protein comprising a target protein fused to a peptide ligand, and a second fusion protein comprising a protein-protein interaction domain (PPID), a self-assembling protein, and a low complexity or intrinsically disordered protein region (IDR), wherein the peptide ligand is capable of binding to the at least one PPID. In some embodiments, the second fusion protein further comprises a fluorescent tag.

Provided herein is a cell expressing the composition comprising a first fusion protein and at least one additional fusion protein as described herein. In some embodiments, the cell is a human cell, an animal cell, or a yeast cell. The nucleic acid construct encoding the target protein (e.g., fusion protein or recombinant protein of interest) can be introduced into living cells. Cells can be grown and protein production can be induced until cells reach desirable density. Cells can then be lysed and if photo-activatable or deactivatable constructs are utilized, the lysate can be, e.g., centrifuged to remove larger cell debris. If photo-activatable or deactivatable constructs are utilized, a supernatant can then be exposed to at least one wavelength of light that the light-sensitive receptor proteins are responsive to, which induces molecules previously within the living cell to cluster or uncluster. In some embodiments, the wavelength of light is predetermined, based on the specific wavelengths to which the light-sensitive receptor protein utilized in the constructs is responsive. Clustering by photoactivation may be applied prior and during the cell lysis step, during which both self-assembling proteins and target fusion proteins are still highly concentrated inside the cells. The induced clusters may then be separated, typically via centrifuge or using a magnetic field, in order to remove, e.g., the unclustered phase.

According to the present disclosure, protein-peptide ligand conjugates are recruited into the phase separated environment generated by the self-assembling low complexity sequence (LCS) or IDR modified cores. Proteins (e.g., enzymes) may be recruited to the phase separated environment through interactions mediated through, e.g., fusion with peptides/proteins that promote interactions with components of the condensed phase.

In some embodiments, the compositions of the present disclosure (e.g., Corelets) form a phase-separated protein clusters or condensates when expressed in a cell. In some embodiments, a concentration of compositions (e.g., Corelets) in a cell can range from about 0.1 mM to about 1000 mM. For example, a composition described herein can be present in a cell in a range of from about 0.1 mM to about 500 mM; from about 5 mM to about 1000 mM, from about 5 mM to about 500 mM, from about 5 mM to about 250 mM, from about 5 mM to about 100 mM, from about 5 mM to about 50 mM, from about 10 mM to about 50 mM, from about 10 mM to about 30 mM, from about 50 mM to about 250 mM, from about 100 mM to about 200 mM, from about 1 mM to about 50 mM, from about 50 mM to about 100 mM, from about 100 mM to about 150 mM, from about 150 mM to about 200 mM, from about 200 mM to about 250 mM, from about 250 mM to about 300 mM, from about 300 mM to about 350 mM, from about 350 mM to about 400 mM, from about 400 mM to about 450 mM, from about 450 mM to about 500 mM, from about 500 mM to about 550 mM, from about 550 mM to about 600 mM, from about 600 mM to about 650 mM, from about 650 mM to about 700 mM, from about 700 mM to about 750 mM, from about 750 mM to about 800 mM, from about 800 mM to about 850 mM, from about 850 mM to about 900 mM, from about 900 mM to about 950 mM, or from about 950 mM to about 1000 mM.

In some embodiments, a concentration of compositions (e.g., Corelets) in a cell can be at least about 0.05 μM, at least about 0.1 μM, at least about 0.2 μM, at least about 0.3 μM, at least about 0.4 μM, at least about 0.5 μM, at least about 1 μM, at least about 2 μM, at least about 3 μM, at least about 4 μM, at least about 5 μM, at least about 10 μM, at least about 15 μM, at least about 20 μM, at least about 25 μM, at least about 30 μM, at least about 40 μM, at least about 50 μM, at least about 60 μM, at least about 70 μM, at least about 80 μM, at least about 90 μM, at least about 100 μM, at least about 200 μM, at least about 300 μM, at least about 400 μM, at least about 500 μM, at least about 600 μM, at least about 700 μM, at least about 800 μM, at least about 900 μM, at least about 1000 μM or more. In some embodiments, a concentration of compositions in a cell can be from 0.1 μM to about 0.1 mM.

A composition (e.g., Corelet) described herein can be present in a cell in an amount of about 1 mM, about 2 mM, about 3 mM, about 4 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 125 mM, about 150 mM, about 175 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1000 mM. In some cases, a composition (e.g., Corelet) described herein can be present in a cell in an amount of about 0.05 μM, about 0.1 μM, about 0.2 μM, about 0.3 μM, about 0.4 μM, about 0.5 μM, about 1 μM, about 2 μM, about 3 μM, about 4 μM, about 5 μM, about 10 μM, about 15 μM, about 20 μM, about 25 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 230 μM, about 400 μM, about 450 μM, about 500 μM, about 550 μM, 600 μM, about 650 μM, about 700 μM, about 750 μM, about 800 μM, about 850 μM, about 900 μM, about 950 μM, or about 1000 μM.

In some embodiments, the particle size of a composition (e.g., Corelets) in a cell can range from about 0.1 nm to about 1000 nm in diameter. For example, a composition described herein can be about 0.1 nm to about 500 nm; from about 5 nm to about 1000 nm, from about 5 nm to about 500 nm, from about 5 nm to about 250 nm, from about 5 nm to about 100 nm, from about 5 nm to about 50 nm, from about 10 nm to about 50 nm, from about 10 nm to about 30 nm, from about 50 nm to about 250 nm, from about 100 nm to about 200 nm, from about 1 nm to about 50 nm, from about 50 nm to about 100 nm, from about 100 nm to about 150 nm, from about 150 nm to about 200 nm, from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, from about 750 nm to about 800 nm, from about 800 nm to about 850 nm, from about 850 nm to about 900 nm, from about 900 nm to about 950 nm, or from about 950 nm to about 1000 nm in diameter.

For example, a composition described herein can be about 1 nm, about 2 nm, about 3 nm, about 4 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 100 nm, about 125 nm, about 150 nm, about 175 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm in diameter.

### Light-Sensitive Receptor Proteins

The at least one light-sensitive receptor protein may comprise one or more similar or different proteins responsive to (i.e., is activated by) at least one wavelength of light, e.g., a wavelength of light in the near ultraviolet (UV), visible or infra-red (IR) regions, which are from about 350 nm to about 800 nm. For example, a light-sensitive receptor protein is responsive to (i.e., is activated by) a light at a wavelength of about 350 nm, 4000 nm, 450 nm, 500 nm, 550 nm, 600 nm, 650 nm, 700 nm, 750 nm, or 800 nm.

In some embodiments, the light-sensitive receptor protein is the engineered improved light-inducible dimer (iLID), that can be activated in specific regions of a cell or an organism using light in a reversible manner. In some embodiments, iLiD comprises a light-oxygen voltage (LOV) domain. In some embodiments, iLID comprises a modified LOV2 domain fused at its C terminus to an ssrA peptide. In some embodiments, the self-assembling protein subunit is fused to two or more light-oxygen voltage (LOV2-ssrA proteins. In other embodiments, other light-sensitive receptor proteins may be utilized, including Cry2, PhyB or a LOV2 domain fused to a signaling peptide other than ssrA.

In some embodiments, light-inducible protein-protein interactions allows for spatial and temporal control that enables formulation of molecular clusters upon light-induced activation. In some embodiments, light-inducible proteins show a change in binding affinity upon light stimulation. For example, a bacterial SsrA peptide may be embedded in the C-terminal helix of a naturally occurring photoswitch, the light-oxygen-voltage 2 (LOV2) domain from Avena sativa. In the dark the SsrA peptide is sterically blocked from binding its natural binding partner, SspB. When activated with, e.g., blue light, the C-terminal helix of the LOV2 domain undocks from the protein, allowing the SsrA peptide to bind SspB.

Any iLIDs may be used in the presently disclosed platform. In some embodiments, a light-inducible protein pair is cryptochrome 2 (Cry2) and CIB1 from, e.g., Arabidopsis thaliana. The Cry2/CIB1 pair shows blue light induced dimerization in both yeast and mammalian cell culture. Another suitable dimerization pair is phytochrome B (PhyB) and PIF, e.g., from A. thaliana. PhyB and PIF interact after irradiation with red light and dissociate with exposure to far-red light. Alternatively, tunable light-controlled interacting protein tags (TULIPs) which make use of the blue light-sensing light-oxygen-voltage (LOV) domain and an engineered PDZ domain may be used. Subcellular localization has been shown with TULIPs in both yeast and mammalian cells.

The cognate partner of the light-sensitive receptor protein can be any appropriate cognate of the light-sensitive receptor protein, which may include but is not limited to ssrB, Zdk, CIB, or PIF for LOV2-ssrA, LOV2, Cry2, or PhyB respectively. In some embodiments, the second fusion protein comprises an IDR, which include but not limited to full length or truncated forms of FUS [SEQ ID NO.: 1], DDX4 [SEQ ID NO.: 2], and hnRNPA1 [SEQ ID NO.: 3]. In some embodiments, the IDR comprises amino acids 1-214 of FUS, 1-236 of DDX4, or 186-320 of HNRNPA1.

In other embodiments, a Dronpa protein, derived from Pectiniidae corals, with intrinsic optogenetic utility in its monomeric state, can be used to form light-dependent dimers and tetramers. In other embodiments, PixELLS system may be used. The PixELL system allows for inverted light control over protein condensate formation. By fusing PixE and PixD with FUSN IDRs, liquid protein condensates may be formed in the dark and dispersed in blue light.

### Self-Assembling Proteins

The self-assembling protein subunit can be any protein that self-assembles, including but not limited to ferritin light chains, ferritin heavy chains, glutamine synthetase, and viral capsid structure proteins, or synthetic engineered self-assembling proteins. Other non-limiting examples of self-assembling proteins according to the present disclosure include a polyhedron-forming protein, a coiled-coil forming protein, a supramolecular self-assembly protein, and a protein oligomer. Non-limiting examples of a polyhedron-forming protein that may be used include: I3-01, O3-33, ATC-HL3, and 3VDX. Non-limiting examples of a coiled-coil forming protein include HexCoil-Ala, 5H2L_2, EE, and RR. Non-limiting examples of a supramolecular self-assembly protein that can be used are: 2AN9 and 1M3U. Non-limiting examples of a protein oligomer that can be used are: 5L6HC3_1 and 2L8HC4_15. A skilled artisan may recognize other self-assembly proteins suitable for use in certain compositions and methods disclosed herein.

In some embodiments, the self-assembling protein is ferritin. In some embodiments, the self-assembling protein is ferritin heavy chain. In some embodiments, the self-assembling protein is ferritin light chain. In some embodiments, the self-assembling protein utilizes ferritin heavy chain subunits, which are capable of self-assembly into a 24 mer complex with a spherical shell structure. In some embodiments, the self-assembling protein utilizes ferritin heavy chain subunits, which are capable of self-assembly into a 48 mer complex with a spherical shell structure. In some embodiments, the self-assembling protein utilizes ferritin heavy chain subunits, which are capable of self-assembly into a 72 mer complex with a spherical shell structure. In some embodiments, the self-assembling protein utilizes ferritin heavy chain subunits, which are capable of self-assembly into a 96 mer complex with a spherical shell structure. Assembled ferritin form deposits of iron-oxide at its internal cavity. By performing certain mutations, such deposits can become ferrimagnetic, thereby making modified ferritin responsive to magnetic field.

In some embodiments, proteins can self-assemble into crystals, filaments, gels, and/or other amorphous aggregates.

In some embodiments, self-assembling comprises at least one oligomerization domain. The oligomerization domain can form interactions that are stable (e.g., long-lived). An oligomer can comprise multiple interacting monomers. For example, an oligomer can comprise 2, 3, 4, 5, 6, 7, 8, 9, 10, 11, 12, 13, 14, 15, 16, 17, 18, 19, 20, 21, 22, 23, 24 or 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 monomers, or even more.

In some embodiments, the particle size of a self-assembling protein can range from about 0.1 nm to about 1000 nm in diameter. For example, a self-assembling protein described herein can be about 0.1 nm to about 500 nm; from about 5 nm to about 1000 nm, from about 5 nm to about 500 nm, from about 5 nm to about 250 nm, from about 5 nm to about 100 nm, from about 5 nm to about 50 nm, from about 10 nm to about 50 nm, from about 10 nm to about 30 nm, from about 50 nm to about 250 nm, from about 100 nm to about 200 nm, from about 1 nm to about 50 nm, from about 50 nm to about 100 nm, from about 100 nm to about 150 nm, from about 150 nm to about 200 nm, from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, from about 750 nm to about 800 nm, from about 800 nm to about 850 nm, from about 850 nm to about 900 nm, from about 900 nm to about 950 nm, or from about 950 nm to about 1000 nm in diameter.

For example, a self-assembling protein described herein can be about 1 nm, about 2 nm, about 3 nm, about 4 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 100 nm, about 125 nm, about 150 nm, about 175 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm in diameter.

### PPIDs

The protein peptide interaction domain (PPID) can comprise peptide recognition segments that are able to recruit proteins (e.g., enzymes) tagged with a corresponding peptide(s) recognized by the PPIDs. Non-limiting examples of PPIDs include a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

In some embodiments, the peptide ligand is a peptide capable of binding to a PPID selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

The PPID may be fused to any portion of the fusion protein. In some embodiments, PPIDs are fused to the N-terminus of the fusion proteins. In some embodiments, PPIDs are fused to the C-terminus of the fusion proteins.

In some embodiment, a PPID is fused to the light-sensitive receptor protein. In some embodiments, a PPID is fused to a cognate partner of a light-sensitive receptor protein. In some embodiment, a PPID is fused to the low complexity or intrinsically disordered protein region (IDR). In some embodiments, a PPID is fused to the self-assembling protein. In some embodiments, a PPID is fused to a fluorescent tag.

In some embodiments, the compositions of the present disclosure utilizes multiple PPIDs and corresponding peptide ligands. In some embodiments, the compositions incorporate 2 PPIDs. In some embodiments, the compositions incorporate 3 PPIDs. In some embodiments, the compositions incorporate 4 PPIDs. In some embodiments, the compositions incorporate 5 PPIDs. In some embodiments, the compositions incorporate 6 PPIDs. In some embodiments, the compositions incorporate 7 PPIDs. In some embodiments, the compositions incorporate 2 PPIDs. In some embodiments, the compositions incorporate 8 PPIDs. In some embodiments, the compositions incorporate 9 PPIDs. In some embodiments, the compositions incorporate 10 PPIDs or more.

### Protein-Peptide Fusion Proteins

In other embodiments, provided herein are engineered fusion proteins comprising peptide ligands fused to a protein of interest. The peptide ligand may be fused to the N-terminus of the protein. Alternatively, the peptide ligand may be fused to the C-terminus of the protein. FIG. 1, 2D depict schematic presentations of C-terminus and N-terminus peptide-tagged protein (designated herein interchangeably as “Cargo”).

In some embodiments, the peptide ligand can comprise from 6 to 50 amino acids, 6 to 45 amino acids, 6 to 40 amino acids, 6 to 35 amino acids, 6 to 30 amino acids, 6 to 25 amino acids, 6 to 20 amino acids, 8 to 50 amino acids, 8 to 45 amino acids, 8 to 40 amino acids, 8 to 35 amino acids, 8 to 30 amino acids, 8 to 25 amino acids, 8 to 20 amino acids, 14 to 50 amino acids, 14 to 45 amino acids, 14 to 40 amino acids, 14 to 35 amino acids, 14 to 30 amino acids, 14 to 27 amino acids, 14 to 25 amino acids, or 14 to 20 amino acids. In some embodiments, at least one of the one or more neo-antigenic peptides comprises from 14 to 30 amino acids. In some embodiments, the peptides is less than 15 amino acids or less in length, from 7 to 11 amino acids in length, or from 8 to 10 amino acids in length.

A wide variety of target proteins may be used in the compositions and methods of the present disclosure. In some embodiments, the protein can be an enzyme. In some embodiments, the protein can be an enzyme implicated in a metabolic pathway such as, but not limited to, carbohydrate metabolism, cellular respiration, cell signaling, amino acid metabolism, vitamin or cofactor metabolism, nucleotide or protein metabolism, lipid metabolism, and the like. Non-limiting examples of metabolic pathways include glycosylation, proteolysis, shikimate pathway, MVA pathway, gluconeogenesis, steroidogenesis, fatty acid synthesis, fatty acid elongation, beta oxidation, peroxisomal beta oxidation, glycoxylate cycle, citric acid cycle, urea cycle, phosphorylation, oxidative phosphorylation, amino acid deamination, lipolysis, lipogenesis, lipolysis, pentose phosphate pathway, carbon fixation, photo respiration, pyruvate decarboxylation, fermentation, and the like. Any enzyme involved in one or more of these and other metabolic pathways and/or genetic material encoding such enzyme may be used in the compositions and constructs of the present disclosure.

In some embodiments, a target protein is a protein is implicated in a disease or condition. In some embodiments, the protein is an antibody or fragment thereof (e.g., adalimumab, rituximab, trastuzumab, bevacizumab, infliximab, or ranibizumab). In some embodiments, the protein is an enzyme (e.g., a therapeutic enzyme such as alpha-galactosidase A, alpha-L-iduronidase, N-acetylgalactosamine-4-sulfatase, dornase alfa, glucocerebrosidase, tissue plasminogen activator, rasburicase, an industrial enzyme (e.g., a catalase, a cellulase, a laccase, a glutaminase, or a glycosidase). In some embodiments, the protein is a biocatalyst (e.g., a transaminase, a cytochrome P450, a kinase, a phosphorylase, or an isomerase)). In some embodiments, the protein is a regulatory protein (e.g., a transcription factor (e.g. Mxr1, Adr1)). In some embodiments, the protein is a peptide hormone (e.g., insulin, insulin-like growth factor 1, granulocyte colony-stimulating factor, follicle-stimulating hormone, or a growth hormone such as human growth hormone). In some embodiments, the protein is a blood clotting protein (e.g., Factor VII). In some embodiments, the protein is a cytokine (e.g., an interferon or erythropoietin). In some embodiments, the protein is a cytokine inhibitor (e.g., interleukin-1 receptor antagonist, soluble IL-1 receptor, soluble TNF-alpha receptors, and certain cytokines, such as IL-4, TGF beta, and IL-10). In some embodiments, the protein is an immunomodulatory protein (e.g., checkpoint inhibitors such as PD-1, or PD-L1, or any protein that modulates the activity thereof such as PD-1 or PD-L1 antibody). In some embodiments, the protein is a tumor suppressor protein. It is appreciated by a skilled artisan that additional proteins or peptides may be used in the modular platform of the disclosure, as desired.

### Fluorescent Tags

The optional fluorescent tag can comprise any appropriate fluorescent protein tag, such as mCherry, although the use of other fluorescent proteins is also envisioned, including but not limited to Green Fluorescent Protein (GFP) and GFP variants, enhanced GFP (EGFP), Cyan Fluorescent Protein (CFP), Yellow Fluorescent Protein (YFP), Red Fluorescent Protein (RFP), Orange Fluorescent Protein (OFP), blue fluorescent protein (BFP), tetracysteine fluorescent motif, or any combination thereof.

The green fluorescent protein may be EGFP (enhanced green fluorescent protein), Emerald (Tsien, Annu. Rev. Biochem., 67: 509-544, 1998), Superfolder (Pedelacq et al., Nat. Biotech., 24: 79-88, 2006), GFP (Prendergast et al., Biochem., 17 (17): 3448-3453, 1978), Azami Green (Karasawa, et al., J. Biol. Chem., 278: 34167-34171, 2003), TagGFP (Evrogen, Russia), TurboGFP (Shagin et al., Mol. Biol. Evol., 21 (5): 841-850, 2004), ZsGreen (Matz et al., Nat. Biotechnol., 17: 969-973, 1999) or T-Sapphire (Zapata-Hommer et al, BMC Biotechnol., 3:5, 2003).

The yellow fluorescent protein may be EYFP (enhanced yellow fluorescent protein, Tsien, Annu. Rev. Biochem., 67: 509-544, 1998), Topaz (Hat et al., Ann. NY Acad. Sci., 1: 627-633, 2002), Venus (Nagai et al., Nat. Biotechnol., 20(1): 87-90, 2002), mCitrine (Griesbeck et al., J. Biol. Chem., 276: 29188-29194, 2001), Ypet (Nguyet and Daugherty, Nat. Biotechnol., 23(3): 355-360, 2005), TagYFP (Evrogen, Russia), PhiYFP (Shagin et al., Mol. Biol. Evol., 21(5): 841-850, 2004), ZsYellow1 (Matz et al., Nat. Biotechnol., 17: 969-973, 1999), or mBanana (Shaner et al., Nat. Biotechnol., 22: 1567-1572, 2004).

The red fluorescent protein may be mRuby (Kredel et al., PLoS ONE, 4(2): e4391, 2009), mApple (Shaner et al., Nat. Methods, 5(6): 545-551, 2008), mStrawberry (Shaner et al., Nat. Biotechnol., 22: 1567-1572, 2004) and AsRed2 (Shanner et al., Nat. Biotechnol., 22: 1567-1572, 2004) or mRFP (Campbell et al., Proc. Natl. Acad. Sci. USA, 99(12): 7877-7882, 2002), RFP-T mRuby2, or TagRFP-T.

The orange fluorescent protein may be Kusabira Orange (Karawawa et al., Biochem. J. 381(Pt 1): 307-312, 2004), Kusabira Orange2 (MBL International Corp., Japan), mOrange (Shaner et al., Nat. Biotechnol., 22: 1567-1572, 2004), mOrange2 (Shaner et al., Nat. Biotechnol., 22: 1567-1572, 2004), dTomato (Shaner et al., Nat. Biotechnol., 22: 1567-1572, 2004), dTomato-Tandem (Shaner et al., Nat. Biotechnol., 22: 1567-1572, 2004), TagRFP (Merzlyak et al., Nat. Methods, 4(7): 555-557, 2007), TagRFP-T (Shaner et al., Nat. Methods, 5(6): 545-551, 2008), DsRed (Baird et al., Proc. Natl. Acad. Sci. USA, 97: 11984-11989, 1999), DsRed2 (Clontech, USA), DsRed-Express (Clontech, USA), DsRed-Monomer (Clontech, USA), or mTangerine (Shaner et al., Nat Biotechnol, 22: 1567-1572, 2004 above). The cyan fluorescent protein may be ECFP (enhanced cyan fluorescent protein, Cubitt et al., Trends Biochem. Sci., 20: 448-455, 1995), mECFP (Ai et al., Biochem. J., 400(3): 531-540, 2006), mCerulean (Koushik et al., Biophys. J., 91(12): L99-L101, 2006), CyPet (Nguyet and Daugherty, Nat. Biotechnol., 23 (3): 355-360, 2005), AmCyan1 (Matz et al., Nat. Biotechnol., 17: 969-973, 1999), Midori-Ishi Cyan (Karawawa et al., Biochem. J., 381(Pt 1): 307-312, 2004), TagCFP (Evrogen, Russia) or mTFP1, (Ai et al, Biochem. J., 400 (3): 531-540, 2006).

The blue fluorescent protein may be EBFP (enhanced blue fluorescent protein, Clontech, USA), EBFP2 (Ai et al., Biochemistry, 46 (20): 5904-5910. 2007), Azurite (Mena et al., Nat. Biotechnol., 24: 1569-1571, 2006) mTagBFP, mTag-BFP2, (Subach et al., Chem. Biol., 15(10): 1116-1124, 2008). The far red fluorescent protein may be mPlum (Wang et al., Proc. Natl. Acad. Sci. USA, 101: 16745-16749, 2004), mCherry (Shanner et al., Nat. Biotechnol., 22: 1567-1572, 2004), dKeima-Tandem (Kogure et al., Methods, 45(3): 223-226, 2008), JRed (Shagin et al., Mol. Biol. Evol., 21(5): 841-850, 2004), mRaspberry (Shanner et al., Nat. Biotechnol., 22: 1567-1572, 2004), HcRed1 (Fradkov et al., Biochem. J., 368(Pt 1): 17-21, 2002), HcRed-Tandem (Fradkov et al., Nat. Biotechnol., 22(3): 289-296, 2004), AQ143 (Shkrob et al., Biochem. J., 392: 649-654, 2005).

### Nucleic Acid Constructs Encoding Corelet Systems

Provided herein is an engineered system comprising a plurality of nucleic acids encoding the fusion proteins as described herein.

Provided herein is an engineered system comprising a first nucleic acid construct encoding a target protein fused to a peptide ligand; and at least one additional nucleic acid construct comprising: (a) a second nucleic acid construct encoding a self-assembling protein and at least one protein-peptide interaction domain (PPID); or (b) a second nucleic acid construct encoding a self-assembling protein, and a third nucleic acid construct encoding a low complexity or intrinsically disordered protein region (IDR), wherein either the second nucleic acid construct or third nucleic acid construct further encode at least one PPID; wherein, when expressed in a cell, the peptide ligand is capable of binding to the at least one PPID.

In some embodiments, the peptide ligand binds to the PPID, thereby recruiting the target protein to the phase-separated clusters. In some embodiments, the system according to the present disclosure is expressed in a cell, wherein at least one additional fusion protein is configured to form an assembled phase, the assembled phase comprising at least one aggregate. In some embodiments, the aggregate comprises phase-separated clusters. In some embodiments, the at least one additional construct further encodes at least one fluorescent tag. In some embodiments, the system comprises a plurality of first constructs.

Provided herein is an engineered system comprising a first nucleic acid construct encoding a target protein fused to a peptide ligand; a second nucleic acid construct encoding a self-assembling protein, and a third nucleic acid construct encoding a low complexity or intrinsically disordered protein region (IDR), wherein either the second nucleic acid construct or third nucleic acid construct further encode at least one PPID; wherein, when expressed in a cell, the peptide ligand is capable of binding to the at least one PPID.

Provided herein is an engineered system comprising a first nucleic acid construct encoding a target protein fused to a peptide ligand; a second nucleic acid construct encoding a self-assembling protein and a light-sensitive receptor protein; and a third nucleic acid construct encodes a low complexity or intrinsically disordered protein region, and a cognate partner to the light-sensitive receptor protein; wherein, when expressed in a cell, the peptide ligand is capable of binding to the at least one PPID. In some embodiments, the second nucleic acid construct encodes the PPID. In some embodiments, the third nucleic acid construct encodes the PPID.

In some embodiments, the light-sensitive receptor protein is iLID. In some embodiments, the cognate partner to the light-sensitive receptor protein is sspB. In some embodiments, the light-sensitive receptor protein is sensitive to at least one visible, ultraviolet (UV) or infrared (IR) wavelength of light.

In some embodiments, the system is expressed in a cell, and the cognate partner of the light-sensitive receptor protein is configured to bind to the light-sensitive receptor protein when the system is irradiated with at least one wavelength of light.

In some embodiments, the second nucleic acid construct and/or the third nucleic acid construct further encodes a fluorescent tag. In some embodiments, the second nucleic acid construct encodes a first fluorescent tag fused to the light-sensitive receptor protein, the self-assembling protein and the PPID; and the third nucleic acid construct encodes a second fluorescent tag fused to the low complexity or intrinsically disordered protein region and the cognate partner to the light-sensitive receptor protein. In some embodiments, the second nucleic acid construct encodes a first fluorescent tag fused to the light-sensitive receptor protein and the self-assembling protein; and the third nucleic acid construct encodes a second fluorescent tag fused to the low complexity or intrinsically disordered protein region (IDR), the cognate partner to the light-sensitive receptor protein, and the PPID.

Provided herein is an engineered system comprising a first nucleic acid construct encoding a target protein fused to a peptide ligand; a second nucleic acid construct encoding a self-assembling protein and at least one PPID; and a third nucleic acid construct encoding a low complexity or intrinsically disordered protein region (IDR), and a self-assembling protein wherein, when expressed in a cell, the peptide ligand is capable of binding to the at least one PPID.

In some embodiments, the second nucleic acid construct and/or the third nucleic acid construct further encodes a fluorescent tag. In some embodiments, the second nucleic acid construct encodes a first fluorescent tag fused to the self-assembling protein and the at least one PPID; and the third nucleic acid construct encodes a second fluorescent tag fused to the low complexity or intrinsically disordered protein region and the self-assembling protein.

Provided herein is an engineered system comprising a first nucleic acid construct encoding a target protein fused to a peptide ligand; and a second nucleic acid construct encoding a PPID and the self-assembling protein, wherein, when expressed in a cell, the peptide ligand is capable of binding to the at least one PPID. In accordance with this embodiment, the target protein is preferably a self-interacting protein. In some embodiments, a self-interacting protein can form dimers. In some embodiments, a self-interacting protein can form trimers. In some embodiments, a self-interacting protein can form tetramers In some embodiments, the second nucleic acid construct further encodes a fluorescent tag.

Provided herein is an engineered system comprising a first nucleic acid construct encoding a target protein fused to a peptide ligand; and a second nucleic acid construct encoding a PPID, ae self-assembling protein, and the low complexity or intrinsically disordered protein region (IDR), wherein, when expressed in a cell, the peptide ligand is capable of binding to the at least one PPID. In some embodiments, the second nucleic acid construct further encodes a fluorescent tag.

Provided is a system a first nucleic acid construct and at least one additional nucleic acid construct as described herein, for use in recruiting the target protein to the at least one additional fusion protein.

Provided is a system a first nucleic acid construct and at least one additional nucleic acid construct as described herein, for use in enhancing a biosynthetic reaction by increasing a local concentration of the target protein, wherein the target protein is one or more enzymes of a metabolic pathway.

In some embodiments, the first nucleic acid construct or the at least one additional nucleic acid construct further comprises a promoter. In some embodiments, the first nucleic acid construct or the at least one additional nucleic acid construct further comprises a sequence encoding a polyadenylation tail. In some embodiments, the first nucleic acid construct or the at least one additional nucleic acid construct further comprises an origin of replication. In some embodiments, the first nucleic acid construct or the at least one additional nucleic acid construct comprises a sequence encoding a 5′ untranslated region (5′-UTR). In some embodiments, the first nucleic acid construct or the at least one additional nucleic acid construct comprises a sequence encoding a 3′ untranslated region (3′-UTR).

Provided herein are nucleic acid constructs encoding peptide ligands fused to proteins of interest. In some embodiments, constructs are designed with conserved recognition sites (for example, for the NotI , XhoI, SpeI, EcoRI, BamHI, HinFI or XbaI restriction enzymes), so that any genetic material that is likewise flanked, in-frame, with the same recognition sites on their 5′ and 3′ ends respectively can be easily incorporated into these expression vectors via ligation reactions.

In some embodiments, the first construct or the at least one additional construct further comprises a restriction site. In some embodiments, the restriction site is a NotI restriction enzyme cutting site. In some embodiments, the restriction site is a XhoI restriction enzyme cutting site. In some embodiments, the restriction site is SpeI restriction enzyme cutting site. In some embodiments, the restriction site is EcoRI restriction enzyme cutting site. In some embodiments, the restriction site is BamHI, restriction enzyme cutting site. In some embodiments, the restriction site is HinFI restriction enzyme cutting site. In some embodiments, the restriction site is XbaI restriction enzyme cutting site. In some embodiments, the restriction site is NheI restriction enzyme cutting site. In some embodiments, the restriction site is MreI restriction enzyme cutting site. In some embodiments, the restriction site is XmaI restriction enzyme cutting site. In some embodiments, the restriction site is AgeI restriction enzyme cutting site. In some embodiments, the restriction site is BspEI I restriction enzyme cutting site. In some embodiments, the restriction site is PacI restriction enzyme cutting site. In some embodiments, the restriction site is PmeI restriction enzyme cutting site. In some embodiments, the restriction site is Kpn restriction enzyme cutting site. In some embodiments, the restriction site is SacI restriction enzyme cutting site. In some embodiments, the restriction site is AscI I restriction enzyme cutting site. In some embodiments, combinations of one or more restriction sites may be used.

In some embodiments, nucleic acid constructs can encode a PPIDs at any location in the nucleic acid construct. In some embodiments, a PPID is encoded at the 5′ end of the nucleic acid construct. In some embodiments, a PPID is encoded at the 3′ end of the nucleic acid construct. In some embodiment, a PPID is encoded 5′ to the light-sensitive receptor protein. In some embodiments a PPID is encoded 5′ to the cognate partner of a light-sensitive receptor protein. In some embodiment, a PPID is encoded 5′ to the low complexity or intrinsically disordered protein region (IDR). In some embodiments, a PPID is encoded 5′ to the self-assembling protein. In some embodiments, a PPID is a PPID is encoded 5′ to the fluorescent tag. In some embodiment, a PPID is encoded 3′ to the light-sensitive receptor protein. In some embodiments a PPID is encoded 3′ to the cognate partner of a light-sensitive receptor protein. In some embodiment, a PPID is encoded 3′ to the low complexity or intrinsically disordered protein region (IDR). In some embodiments, a PPID is encoded 3′ to the self-assembling protein. In some embodiments, a PPID is a PPID is encoded 3′ to the fluorescent tag.

### Methods of Aggregation

Further provided in some embodiments are methods for inducing protein aggregation, methods for phase-separation, methods for cluster formation, and/or methods of formation of an assembled state. The methods generally comprises several steps. In the first step, nucleic acids encoding light-sensitive first and second fusion proteins, one of which is conjugated to a PPID, are provided (see, e.g., FIG. 1A-B, 2A-B). Alternatively, a single light insensitive construct encoding self-assembling subunit, a PPID and optionally and IDR is provided (see, e.g., FIG. 1C, 1E, 1F, 2C, 2E, 2F). Furthermore, protein-peptide ligands, or nucleic acid constructs encoding protein-peptide ligands are provided (see, e.g. FIG. 1D, 2D). The fusion proteins are expressed, e.g., in a living cell, under conditions which allow the fusion protein to self-assemble to a core (e.g., a spherical core) comprising self-assembled proteins and/or IDRs. Multimerized IDR-based self-interactions leads to the formations of larger clusters, shown, e .g., in (FIG. 2C), capable of recruiting cargo with the PPIDs.

Formation of self-assembling (Core) or IDRs allows for cargo recruitment, e.g., recruitment of a target protein to phase-separated clusters. Thus, in some embodiments, the present disclosure is a composition comprising a first fusion protein and at least one additional fusion protein as described herein, for use in recruiting the target protein to the at least one additional fusion protein.

Provided herein is a method of forming an assembled phase, the method comprising expressing, within a cell, a composition comprising a first fusion protein and at least one additional fusion protein as described herein, and allowing the composition to undergo phase separation into at least one condensed phase within the living cell. In some embodiments, the at least one condensed phase comprises phase-separated clusters.

Provided herein is a method of recruiting a protein to an assembled phase, the method comprising expressing, within a cell, a composition comprising a first fusion protein comprising a peptide-ligand and a target protein, and at least one additional fusion protein as described herein, and allowing the composition to undergo phase separation into at least one condensed phase within the living cell. The peptide ligand and PPID interact, thereby recruiting the target protein to the assembled phase. In some embodiments, the at least one condensed phase comprises phase-separated clusters.

Provided is a composition comprising a first fusion protein and at least one additional fusion protein as described herein, for use in recruiting the target protein to the at least one additional fusion protein.

As these constructs are modular, properties can be varied as desired by a skilled artisan, including activation/deactivation times, wavelength sensitivity, core size, light-sensitive receptor protein density on the core, IDR sequences, and reversibility.

### Additional Platforms

The disclosed platform can be used to dynamically reconfigure a Corelet system as described herein. The disclosed platform can also be used to reconfigure systems such as optoDroplet and CasDrop systems, and may be used for a variety of applications including genomic targeting, drug discovery etc. An optoDroplet system can involve proteins whose behavior can be altered by exposure to light. Phase transitions can be induced to create membraneless organelles by switching on the light-activated proteins. The transitions can be turned off by simply turning the light off. Increasing the light intensity and protein concentrations can control the transition. The optoDroplet system can comprise two segments fused together, where the first segment is a light-sensitive receptor protein (such as Cry2, Cry2olig, PhyB, PIF, light-oxygen-voltage sensing (LOV) domains, or Dronpa) that is sensitive to at least one wavelength of light, and the second segment can be a low complexity sequence (LCS) or an intrinsically disordered protein region (IDR) (such as those present in FUS, Ddx4, and hnRNPAl), or in some cases a folded protein/domain. The optoDroplet system can function by irradiating the optoDroplet system with a wavelength of light the light-sensitive receptor protein is sensitive to. The optoDroplets then self-assemble while exposed to that light, and the assembly of the LCSs or IDRs can cause phase separation (or droplet formation) to occur. When the light is turned off, this phase separation can be reversed. For example, the optoDroplet system can comprise FUS^{N}-Cry2 forms liquid-like spherical droplets that rapidly exchange monomers in and out of clusters. The fusion protein FUS^{N}-Cry2 can be created by fusion of Cry2 to the N terminal intrinsically disordered region (IDR) from the protein FUS (e.g., FUS^{N}). The CasDrop system can include a first structure having a Cas-based genomic targeting protein fused or attached to one or more sequences, each of which may be a light-sensitive receptor, a chemical-sensitive receptor, a light sensitive oligomerization protein or a non-light sensitive dimerization module. The CasDrop system may also include a second structure, which includes a cognate partner of the light- or chemical-sensitive receptor protein or a dimerization domain complementary to the dimerization module, where the cognate partner or complementary dimerization domain is fused to at least one transcriptional regulatory protein having a full length or truncated low complexity or intrinsically-disordered protein region. The first structure may include dCas9 fused to SunTag, which is attached to another construct having a single chain variable fragment antibody fused to a superfolding variant of GFP (sfGFP) and iLID, where the single chain variable fragment antibody is a cognate for SunTag. The first structure may include at least one reporter protein. The cognate partner or complementary dimerization domain may be fused to full length or truncated BRIM, FUS, or TAF15. The first structure may include repeating sequences. Detailed descriptions of the optoDroplet system and the CasDrop system can be found in US Pub. No. 2017/0355977 and PCT/US2019/014666, respectively, each of which is incorporated herein by reference herein in its entirety.

In other embodiments, phase-separated clusters can be formed from other stimuli. For example, phase-separated clusters form upon exposure to temperature, chemicals, and combination thereof. Examples of chemically-induced aggregates include, but are not limited to, CRISPR-genome organization (CRISPR-GO) systems, e.g., an abscisic acid (ABA) inducible ABI/PYL1 system, and Trimethoprim-Haloligand (TMP-Htag) inducible DHFR/HaloTag system as described in Haifeng Wang H et al., Cell, Vol. 175, Issue 5, pages 1405-1417 (2018). Additional chemically-inducible system include rapamycin-induced protein-based hydrogels as described in Nakamura, H., et al., Nature Mater 17, 79-89 (2018). Examples of temperature-induced aggregates include intrinsically disordered proteins as described herein. The interactions between intrinsically disordered proteins that can be used in such systems are largely thought and shown to be temperature dependent.

In other embodiments, a system is provided comprising: a first DNA construct encoding an enzyme or fluorescent protein fused to a peptide ligand; a second DNA construct encoding a light sensitive protein fused to a fluorescent protein tag, and ferritin heavy chain; and a third DNA construct encoding an IDR fused to a fluorescent protein and a cognate partner to the light sensitive protein, wherein either the second or third DNA construct also encodes at least one PPID fused to either the light sensitive protein or the IDR, wherein the cognate partner is configured to bind to the light sensitive protein when the system is irradiated with at least one wavelength of light, and wherein the peptide ligand is capable of binding to the at least one PPID.

In other embodiments, a system is provided comprising: a first DNA construct encoding an enzyme or fluorescent protein fused to a peptide ligand; and a second DNA construct encoding at least one PPID fused to an IDR, a fluorescent protein tag, and ferritin heavy chain, wherein the peptide ligand is capable of binding to the at least one PPID.

In other embodiments, a system is provided comprising: a first DNA construct encoding an enzyme or fluorescent protein fused to a peptide ligand; and a second DNA construct encoding at least one PPID fused to a fluorescent protein tag and ferritin heavy chain; a third DNA construct encoding an IDR fused to a fluorescent protein tag and ferritin heavy chain; wherein the peptide ligand is capable of binding to the PPID.

In other embodiments, a platform for inducing clustering is provided, the platform comprising: a library comprising a plurality of DNA constructs and the biological organisms they are introduced into to allow for expression of proteins that the DNA encodes for, wherein each DNA constructs is either: a DNA construct that encodes for condensate-forming protein units fused to one or more PPIDs that are able to recruit proteins tagged with at least one corresponding peptide recognized by the one or more PPIDs, the condensate-forming protein units capable of being additionally fused to, or replaced with, other protein domains that make condensate formation inducible by specific stimuli; or a DNA construct adapted to allow for facile tagging of a protein of interest with peptides that interact and recruit to the engineered condensates, the DNA construct being designed with conserved recognition sites, such that any genetic material that is likewise flanked, in-frame, with the same recognition sites on their 5′ and 3′ ends respectively can be incorporated into these expression vectors via ligation reactions.

Provided is a method for inducing clustering, comprising: providing a cell that incorporates a first system as described herein irradiating the cell during fermentation with at least one wavelength of light the light sensitive protein in the first system is sensitive to; and allowing the proteins encoded by the first system to cluster. In some embodiments, n the clustering increases the total production of at least one chemical in the cell as compared to a cell that does not incorporate the first system or that was not irradiated with the at least one wavelength of light.

Provided is a for screening interaction domains, comprising: providing a first system as described herein comprising a first PPID, and a second system as described herein comprising a second PPID; irradiating the first and second system with at least one wavelength of light the light sensitive protein in each system is sensitive to; and quantifying the colocalization of a fluorescent protein in the first DNA construct of each system.

### Uses

Corelet systems described herein can be used for recruiting proteins of interest to an assembled phase for a broad range of uses.

In some embodiments, a protein of interest is an enzyme. In some embodiments, the enzyme is an enzyme of a metabolic pathway. Thus, provided is a composition comprising a first fusion protein and at least one additional fusion protein as described herein, for use in enhancing a biosynthetic reaction by increasing a local concentration of the target protein, wherein the target protein is one or more enzymes of a metabolic pathway.

In other embodiments, provided is a method for increasing production of at least one chemical in a cell, the method comprising the steps of expressing, in the cell, a composition comprising a first fusion protein and at least one additional fusion protein as described herein, under conditions sufficient to form an assembled phase.

In other embodiments, provided is a method for enhancing a biochemical (e.g., biosynthetic) reaction in a cell, the method comprising the steps of expressing, in the cell, a composition comprising a first fusion protein and at least one additional fusion protein as described herein, under conditions sufficient to form an assembled phase.

In other embodiments, provided herein is a method for screening for protein-protein interactions, the method comprising expressing, in a cell, a first composition comprising a first target protein as described herein, and a second composition comprising a second target protein as described herein, under conditions sufficient to form an assembled phase; and measuring colocalization of the first and the second target proteins in the cell.

In other embodiments, provided herein is a method of screening for compounds capable of modulating a protein-protein interaction, the method comprising: expressing, in a cell, a first composition comprising a first target protein as described herein, and a second composition comprising a second target protein as described herein, under conditions sufficient to form an assembled phase; contacting the cell with a test compound; measuring colocalization of the first and the second target proteins in the cell in the presence of the test compound; and comparing colocalization of the first and the second target proteins in the presence of the test compound to a reference sample measured in the absence of the test compound; wherein a change in the colocalization of the first and the second target proteins in the presence of the test compound as compared with the reference sample is indicative of the ability of the test compound to modulate said protein-protein interaction. In some embodiments, the method further comprises administering to a subject the test compound to treat a condition.

In some embodiments, measuring colocalization of the first and the second target proteins comprises quantifying colocalization of a fluorescent protein in each of the first and composition the second composition. In some embodiments, measuring colocalization of the first and the second target proteins comprises detecting location of the first and the second target proteins in the cell. In some embodiments, detecting comprises imaging. In some embodiments, detecting location of the first and the second target proteins in the cell comprises detecting a signal. In some embodiments, the signal is an electronic signal or an electromagnetic signal. In some embodiments, the signal is optically detectable. In some embodiments, the optically detectable signal is a fluorescence signal or a luminescence signal. In some embodiments, the optically detectable signal is a small-molecule dye, a fluorescent molecule or protein, a quantum dot, a colorimetric reagent, a chromogenic molecule or protein, a Raman label, a chromophore, or a combination thereof.

In some embodiments, measuring colocalization of the first and the second target proteins comprises determining presence or amount of a compound in a biological pathway, e.g., a biosynthetic pathway.

Provided herein is a method of screening for protein-nucleic acid interactions, the method comprising: expressing, in a cell, a composition comprising a target protein as describe herein, under conditions sufficient to form an assembled phase; and measuring binding of a nucleic acid to the target protein in the cell.

Provided herein is a method of screening for compounds capable of modulating a protein-nucleic acid interaction, the method comprising: expressing, in a cell, a composition a target protein as described herein, under conditions sufficient to form an assembled phase; contacting the cell with a test compound; measuring binding of a nucleic acid to the target protein in the cell in the presence of the test compound; and comparing binding of the nucleic acid to the target protein in the presence of the test compound to a reference sample measured in the absence of the test compound; wherein a change in the binding of the nucleic acid to the target protein in the presence of the test compound as compared with the reference sample is indicative of the ability of the test compound to modulate protein-nucleic acid interaction.

In some embodiments, measuring binding of the nucleic acid to the target protein comprises detecting a signal from the nucleic acid. In some embodiments, the measuring further comprises, prior to detecting, staining the nucleic acid or binding the nucleic acid with a detectable probe. In some embodiments, the signal is a fluorescence signal or a luminescence signal.

In some embodiments, measuring binding of the nucleic acid to the target protein comprises determining presence or amount of a compound in a biological pathway.

In some embodiments, the compound disrupts binding of the target protein to the nucleic acid. In other embodiments, the compound enhances binding of the target protein to the nucleic acid.

In other embodiments, provided herein is a method of screening for a compound capable of modulating a target protein, the method comprising expressing, in a cell, a composition comprising a first fusion protein and at least one additional fusion protein as described herein, under conditions sufficient to form an assembled phase; contacting the cell with a test compound; measuring a biological parameter of the target protein; and comparing said biological parameter to a reference sample measured in the absence of the test compound; wherein a change in the biological parameter in the presence of the test compound as compared with the reference sample is indicative of the ability of the test compound to modulate said target protein. In some embodiments, the biological parameter is enzymatic activity, metabolism, signaling, transcription, translation, degradation, a post-translational modification, or presence or amount of a compound in a biological pathway.

In some embodiments, modulating the target protein comprises inhibiting the amount of activity of the target protein. In some embodiments, modulating the target protein comprises activating or increasing the amount or activity of the target protein. In some embodiments, the method further comprises the step of administering to a subject the test compound to treat a condition.

In other embodiments, provided is a method of treating a condition or disorder in a subject, the method comprising the step of expressing a composition as comprising a first fusion protein and at least one additional fusion protein as described herein, in a cell of the subject under conditions sufficient for the at least one additional fusion protein to form an assembled phase. In some embodiments, the condition or disorder is a condition or disorder of a metabolic, signaling, a transcription, a translation, degradation pathway.

In other embodiments, provided is a composition as comprising a first fusion protein and at least one additional fusion protein as described herein, for use in treating a condition.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. In some embodiments, the disease or condition implicates one or more target proteins as described herein.

### Kits

In some embodiments, kits may also be provided to simplify the use of these methods.

The kits will generally include plasmids for the fusion proteins as described above, as well as at least one light emitting device that can be used to activate or deactivate the light-sensitive receptor proteins. Kits may also include a microfabricated device for activation and collection of condensed liquid phases.

In an aspect of the present disclosure, a kit is provided for forming light-induced protein nano-clusters, the kit comprising a first expression vector including a polynucleotide encoding a first fusion protein comprising a light-induced heterodimerized protein and a first self-assembled protein; and a second expression vector including a polynucleotide encoding a second fusion protein comprising a cognate partner protein capable of forming a heterodimer with the light-induced heterodimerizing protein, and a low-complexity or intrinsically disordered protein region (IDR), wherein either the first or the second expression vector further includes a polynucleotide encoding a protein-protein interaction domain (PPID). The kit may further include a third expression vector including a polynucleotide encoding a third fusion protein comprising a target protein and a peptide ligand.

In an aspect of the present disclosure, a kit is provided for forming constitutive protein nano-cluster. The kit comprises a first expression vector including a polynucleotide encoding a first fusion protein comprising a self-assembling protein and at least one PPID, and a second expression vector including a polynucleotide encoding a second fusion protein comprising a low complexity or intrinsically disordered protein region (IDR), and a self-assembling protein. The kit may further include a third expression vector including a polynucleotide encoding a third fusion protein comprising a target protein and a peptide ligand.

In an aspect of the present disclosure, a kit is provided for forming constitutive protein nano-clusters comprising a first expression vector including a polynucleotide encoding a first fusion protein comprising a PPID and a self-assembling protein. The kit may further include a second expression vector including a polynucleotide encoding a second fusion protein comprising a self-interacting target protein and a peptide ligand.

In an aspect of the present disclosure, a kit is provided for forming constitutive protein nano-clusters. The kit comprises a first expression vector including a polynucleotide encoding a first fusion protein comprising a PPID, a self-assembling protein, and a low complexity or intrinsically disordered protein region (IDR). The kit may further include a second expression vector including a polynucleotide encoding a second fusion protein comprising a target protein and a peptide ligand.

### Expression Systems

The construct described herein can be ribonucleic acid, deoxyribonucleic acid, or a combination thereof. The construct described herein can be an expression vector. The expression vector can be a recombinant vector comprising operably linked polynucleotide elements that facilitate and optimize expression of a gene of interest in a particular host organism (e.g., a bacterial expression vector, a yeast expression vector, or mammalian expression vector). Polynucleotide sequences that facilitate gene expression can include, for example, promoters, enhancers, transcription termination sequences, and ribosome binding sites. In the context of an expression vector, the vector can be readily introduced into a host cell, e.g., mammalian, bacterial, yeast.

Methods for delivering vectors/constructs or other nucleic acids (such as in vitro transcribed RNA) into host cells such as bacterial cells, yeast cells and mammalian cells can include electroporation, transformation, transfection or transduction.

For example, methods for delivering vectors or other nucleic acid molecules into bacterial cells can include electroporation and chemical transformation of E. coli cells. The E. coli cells may have been rendered competent by previous treatment with divalent cations such as CaCl₂.

For another example, methods for delivering vectors or other nucleic acid molecules into yeast cells can include electroporation and chemical transformation of the yeast cells. Example methods used in transformation of yeast cells include lithium, electroporation, biolistic and glass bead methods.

Chemical methods for introducing a nucleic acid molecules into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Methods for delivering vectors or other nucleic acid (such as RNA) into mammalian cells can include but are not limited to calcium phosphate precipitation, electroporation, lipid-based methods (liposomes or lipoplexes) such as Transfectamine (Life Technologies) and TransFectin (Bio-Rad Laboratories), cationic polymer transfections, for example using DEAE-dextran, direct nucleic acid injection, biolistic particle injection, and viral transduction using engineered viral carriers (termed transduction, using e.g., engineered herpes simplex virus, adenovirus, adeno-associated virus, vaccinia virus, Sindbis virus), and sonoporation. Any of these methods find use with the present disclosure.

Examples of plasmids used in the present disclosure include, but are not limited to, pMW482, pMW663, pMW011, pMW012, pMW070, pMW072, pMW073, pMW074, pMW477, pMW474, pMW334, pMW479, pMW476, pMW471, pMW470, pKX038, pKX039, pKX044, pKX045, pKX046, pKX053, pKX005, pKX006, pKX052, pKX048, pKX049, pKX050, pKX051, pKX100, pMW748, pMW335, pMW548, pMW551, pMW549, and pMAL857.

While the current set of DNA plasmids used in the disclosed examples are designed for protein expression in yeast, straightforward molecular biology techniques can be employed to insert these components in vectors for expression in other cells types (e.g., human cells) and organisms.

Thus, exemplary devices and systems for nucleated protein clustering have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

### EXAMPLES

### Example 1. Materials and Methods

Corelet constructs originated from Bracha et al. (Bracha, D. et al. Mapping Local and Global Liquid Phase Behavior in Living Cells Using Photo-Oligomerizable Seeds. Cell vol. 175 1467-1480.e13 (2018)) and cloned into the pJLA vector series (Avalos, J. L., Fink, G. R. & Stephanopoulos, G. Compartmentalization of metabolic pathways in yeast mitochondria improves the production of branched-chain alcohols. Nat. Biotechnol. 31, 335-41 (2013)) with NotI and SpeI restriction sites upstream of the coding sequence as a location for PPID insertion. Domains were synthesized (SynBio Technologies) with upstream NotI site and downstream (AG₄S)₂ flexible linker followed by SpeI site, allowing for final gene construction via restriction/ligation cloning.

Constitutive Corelets were constructed from the iLID::GFP::FTH1 construct of Corelets. The FUSn coding sequence was isolated via PCR and was subcloned into the Corelet construct, linearized with SpeI and BamHI, via Gibson assembly. PPID domains were inserted as described above.

The fluorescent protein present in each Corelet construct has been switched for various applications using similar a Gibson assembly approach. Components, such as IDR or fluorescent protein, were occasionally removed from constructs by PCR amplifying the entire plasmid outside of the region to omit; primers were designed with appropriate overhangs for reassembly with Gibson assembly.

GFP cargo is cloned into the pJLA vector series (Avalos, (2013)), whereby the coding sequence is flanked upstream by NotI-KOZAK-NheI and downstream by XhoI-STOP codon. These restriction sites were leveraged to add the PPID ligand tags to the GFP cargo. All such modifications were made via oligo annealing to create the ligand with a linker and corresponding sticky ends for ligation; a (G₄S)₂ flexible linker was used in all cases. For N-terminal ligand tag, the annealed oligos contained a NotI sticky end on the 5′ end followed by KOZAK sequence with start codon prefacing the ligand sequence, which was followed by the linker and NheI sticky end on the 3′ end. For C-terminal ligand tags, XhoI sticky ends were present on both sides of the annealed oligos with the linker on the 5′ end. All Corelet based constructs were subcloned via restriction/ligation into His3 locus integration plasmid, while cargo constructs were similarly subcloned into Leu2 integration plasmid, for chromosomal integration into the yeast genome. All oligonucleotides were synthesized by Integrated DNA technologies and all enzymes used for construct generation are from New England Biolabs. Constructs were sequence verified (Genewiz) before yeast transformation and experimentation.

All yeast are of CEN.PK2-1C origin. All transformations were carried out using standard lithium acetate protocol to produce genomically integrated, stable cells. Cells are grown and fermented in synthetic complete media with 2% glucose. Cells were grown 30° C., shaking at 200 RPM.

All imaging is performed on yeast harvested during exponential growth. Briefly, yeast are grown overnight in selective media. The next day, yeast are inoculated in fresh complete media at an OD₆₀₀ of approximately 0.1-0.5. Following growth for 5-6 hours, cells are verified to be in mid exponential phase, as defined by having OD₆₀₀ in the range of 1.5-6. Cells are diluted to OD₆₀₀ 0.5-1 in PBS; 100 μL of this dilution is added to Concanavalin A (Sigma-Aldrich) treated 96-well glass plate (Cellvis). Cells are treated with Membrite Fix 640/660 membrane dye (Biotium).

For constitutive Corelets, cells are fixed with 4% paraformaldehyde (Electron Microscopy Science), diluted in PBS. Confocal images were collected using a Yokogawa CSU-X1 spinning disk with a 100×, NA 1.49, Apo TIRF oil immersion objective (Nikon) on a Nikon Exlipse Ti body and Andor DU-897 EMCCD camera. Protein fusions containing meGFP (GFP) and mCherry, were imaged using 488 nm and 561 nm wavelength lasers, respectively; membrane dye imaged with 640 nm laser. The 488 nm laser was used to promote iLID-sspB interactions necessary to lead to light-induced Corelet formation. Constitutive Corelets are visualized as a maximum intensity projection from a Z-series of images collected over a 5 micron range centered at the position of largest average cell cross-section and with step size similar to the Z-resolution of the microscope.

To assay cargo recruitment to Corelet-based puncta, Pearson Correlation Coefficients were calculated between pixel-wise intensities from the fluorescent marker for puncta to that for the cargo, on a per-cell basis. In all cases, GFP was used as cargo. For light-induced Corelets, data is shown for PPID fusion to the IDR of the Corelets. A variant was utilized where GFP was removed from the construct; therefore iLID::FTH1 was expressed as a gene cassette with PPID::FUSn::mCherry::SspB, which was coexpressed with each shown GFP-ligand fusion. This set of data did not utilize membrane dye, instead cells were segmented based on mCherry signal in the first frame of imaging. Following 30 seconds of imaging only mCherry (for cell segmentation), the light-induced puncta were induced via imaging of the GFP cargo, sequentially with mCherry, for 9 minutes. The final frame of this 9 minute series was used at the basis for Pearson Correlation Coefficient determination. A minimum of 20 cells from at least 2 fields of view were analyzed to determine a mean value for each cargo-ligand:Corelet-Domain pair.

For similar analysis with the constitutive Corelets, a variant construct was utilized that marked the puncta with mCherry, PPID::FUSn::mCherry::FTH1, which was coexpressed with each shown GFP-ligand fusion. Cells were segmented based membrane dye signal from a confocal image centered on the yeast at the Z-plane of largest yeast cross-section. Maximum intensity projections of the Corelet puncta signal and GFP cargo were used for Pearson Correlation Coefficient calculation. A minimum of 50 cells from at least 2 fields of view were analyzed to determine a mean value for each cargo-ligand:Corelet-Domain pair.

FIG. 2G depicts a time series of yeast cells expressing light-induced Corelets. IDR used is from the human FUS protein, N-terminus (hFUSn). Light was on from 0-6 minutes (underlined); depicted is a cell outline with a membrane dye. FIG. 2H depicts hFUSn Constitutive Corelets, maximum Z-projection; depicted is a cell outline with a membrane dye. FIG. 2I depicts constitutive Corelets without IDRs, recruiting self-interacting (dimeric) proteins with PDZ PPID, maximum Z-projection. Depicted is a cell outline with a membrane dye. All scale bar, 5 μm.

### Example 2. GFP Recruitment to Constitutive Corelets

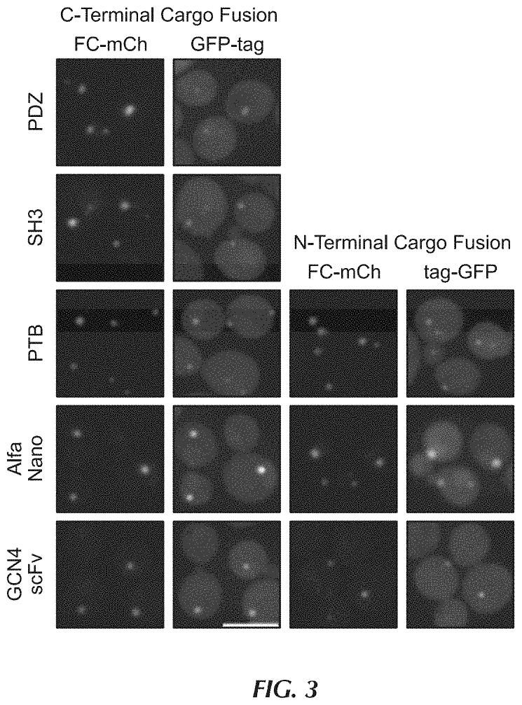

To assay cargo recruitment to Corelet-based puncta, GFP was used as cargo as described in Example 1. FIG. 3 shows GFP cargo recruitment to mCherry (mCh) tagged constitutive Corelet (FC) fused to PPIDs. GFP is tagged with that domain's corresponding peptide as a C-terminal (left) or N-terminal (right) fusion. Scale bar, 5 μm.

### Example 3. Orthogonal Enrichment of Proteins Tagged with Corresponding Peptides

Orthogonality of PPIDs was tested using the system shown in FIG. 2 by co-expressing the clustering components with PPID and a fluorescent protein tagged with corresponding peptides. S. cerevisiae strains were generated to test each PPID-peptide tag combination as described in Example 1. Upon viewing the samples via confocal microscopy after blue-light activation, the degree of colocalization of the tagged fluorescent protein with the marker of the clustering component was assayed via a correlation coefficient.

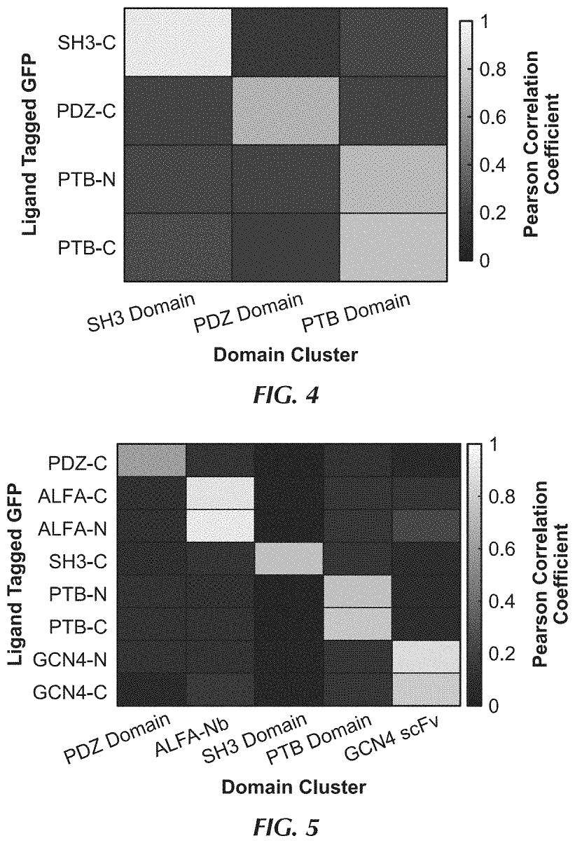

FIG. 4 shows orthogonal enrichment of proteins tagged with corresponding peptides in Light Induced Corelets. Data shown as a heat map of the resulting mean Pearson correlation coefficient for each PPID-peptide pair in this interaction matrix. Peptide tag used shown on the left, and domain in the clustering construct labelled at the bottom.

FIG. 5. shows orthogonal enrichment of proteins tagged with corresponding peptides in Constitutive Corelets. Data shown as a heat map of the resulting mean Pearson correlation coefficient for each PPID-peptide pair in this interaction matrix. Peptide tag used shown on the left, and domain in the clustering construct labelled at the bottom.

### Example 4. Shikimate Biosynthesis

For shikimate production, a background strain was generated which involves knockouts and downregulation of native yeast genes that encode for metabolic reactions that detract from aromatic amino acid production. TKL1, TAL1, ENO2, and ARO4 were clustered to enhance uptake of E4P and PEP into the shikimic acid pathway; ARO4 catalyzes the first step of this pathway. TKL1 and ENO2 were C-terminally tagged with PTB ligand and TAL1 and ARO4 were C-terminally tagged with PDZ ligand. These enzymes were coexpressed in the aforementioned background strain with two copies of the constitutive Corelet construct PPID::FUSn::GFP::FTH1, where PPID=PDZ for one copy, and PTB for the other. Variants were likewise generated where not all enzymes were tagged with the mentioned ligand; in one case TKL1 is not tagged, and in another, both TKL1 and TAL1 are both untagged. The strains are otherwise generated identically. Data shown indicate shikimate production in 48-hour 1 mL high-cell density fermentations. Briefly, cells inoculated from agar plates into 1 mL media in a 24-well plate (CytoOne) and grown for 24 hours; these are diluted 1:1000 in 1 mL fresh media on a new plate and grown for 24 hours; cells are spun down and media is replaced at which point the 48 hour fermentation begins. Afterwards, media is harvested following 45 minute centrifugation, 17,000 RCF, 4° C. for HPLC analysis. Liquid chromatography was performed using an Agilent 1260 Infinity system with a Aminex HPX-87H ion-exchange column (BioRad) maintained at 50° C. and 5 mM sulfuric acid mobile phase (0.5 mL/min flowrate). Shikimate was monitored using diode array detector with 210 nm detection wavelength. Peaks areas were calibrated to concentrations using a calibration curve with shikimate standard (Sigma Aldrich).

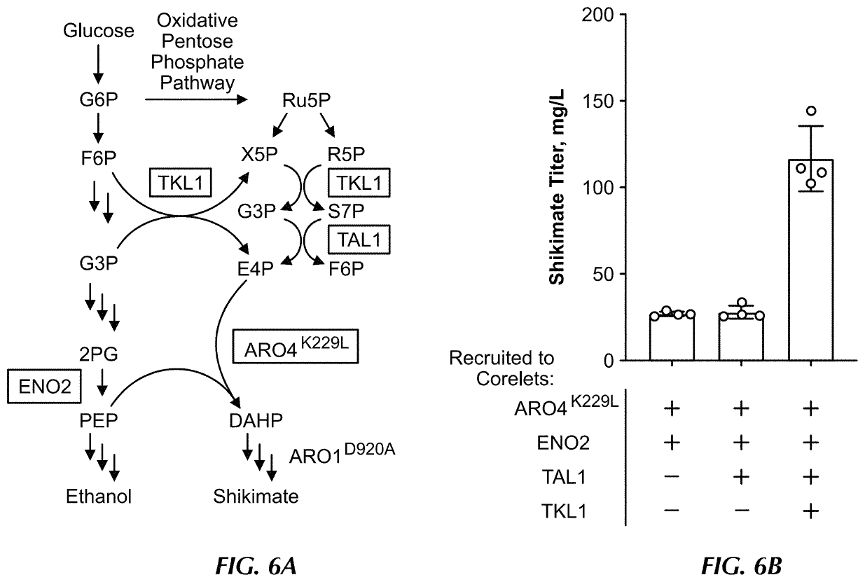

FIG. 6. shows enzyme enrichment with PPIDs in constitutive Corelets increase shikimate production. FIG. 6A: ARO4 condenses PEP, produced by ENO2, and E4P, produced by TKL1 and TAL1, to produce DAHP, which is converted to Shikimate, a notable precursor to natural aromatic amino acids and derivatives of them (AAA). FIG. 6B: TAL1 and ARO4 recruited to clusters with PDZ tag and domain; TKL and ENO2 recruited by PTB tag and domain. The case when all four enzymes are recruited through expression with PTB/PDZ tags (+) shows enhanced shikimate production compared to when certain enzymes are removed from the cluster through expression without fusion to PTB/PDZ tag (−).

The examples and embodiments described herein are for illustrative purposes only and various modifications or changes suggested to persons skilled in the art are to be included within the spirit and purview of this application and scope of the appended claims.

What is claimed is:

1. A composition comprising:
a first fusion protein comprising a target protein fused to a peptide ligand; and
at least one additional fusion protein comprising:
(a) a second fusion protein comprising a self-assembling protein and at least one protein-peptide interaction domain (PPID); or
(b) a second fusion protein comprising a self-assembling protein, and a third fusion protein comprising a low complexity or intrinsically disordered protein region (IDR), wherein either the second fusion protein or the third fusion protein further comprises at least one PPID;
wherein the peptide ligand is capable of binding to the at least one PPID.

2. The composition according to claim 1, wherein the composition is configured to form an assembled phase, the assembled phase comprising at least one aggregate.

3. The composition according to claim 2, wherein the aggregate comprises phase-separated clusters.

4. The composition according to claim 3, wherein the peptide ligand binds to the PPID, thereby recruiting the target protein to the phase-separated clusters.

5. The composition according to claim 3 or 4, wherein the phase-separated clusters form upon exposure of said at least one additional fusion protein a stimulus selected from the group consisting of light, temperature, chemicals, and any combination thereof.

6. The composition according to any one of the preceding claims, wherein the composition is present in a cell.

7. The composition according to claim 6, wherein the composition increases production of at least one chemical in the cell as compared with a cell that does not contain the composition.

8. The composition according to any one of the preceding claims, wherein the target protein is an enzyme.

9. The composition according to claim 8, wherein the enzyme is an enzyme of a metabolic pathway.

10. The composition according to any one of the preceding claims, wherein the target protein is a fluorescent protein.

11. The composition according to any one of the preceding claims, wherein the at least one additional fusion protein further comprises at least one fluorescent tag.

12. The composition according to any one of the preceding claims, comprising a plurality of first fusion proteins.

13. The composition according to any one of the preceding claims wherein the at least one additional fusion protein comprises a second fusion protein comprising the self-assembling protein, and a third fusion protein comprising the low complexity or intrinsically disordered protein region (IDR), wherein either the second fusion protein or the third fusion protein further comprises at least one PPID.

14. The composition according to claim 13, wherein
the second fusion protein comprises the self-assembling protein and a light-sensitive receptor protein; and
the third fusion protein comprises the low complexity or intrinsically disordered protein region, and a cognate partner to the light-sensitive receptor protein.

15. The composition according to claim 14, wherein the PPID is fused to the second fusion protein.

16. The composition according to claim 14, wherein the PPID is fused to the third fusion protein.

17. The composition according to any one of claims 14-16, wherein the light-sensitive receptor protein is iLID.

18. The composition according to any one of claims 14-17, wherein the cognate partner to the light-sensitive receptor protein is sspB.

19. The composition according to any one of claims 14-18, wherein the light-sensitive receptor protein is sensitive to at least one visible, ultraviolet (UV) or infrared (IR) wavelength of light.

20. The composition according to any one of claims 14-19, wherein the cognate partner of the light-sensitive receptor protein is configured to bind to the light-sensitive receptor protein when the system is irradiated with at least one wavelength of light.

21. The composition according to any one of claims 14-20, wherein the second fusion protein and/or the third fusion protein further comprises a fluorescent tag.

22. The composition according to claim 21, wherein
the second fusion protein comprises a first fluorescent tag fused to the light-sensitive receptor protein, the self-assembling protein and the PPID; and
the third fusion protein comprises a second fluorescent tag fused to the low complexity or intrinsically disordered protein region and the cognate partner to the light-sensitive receptor protein.

23. The composition according to claim 21, wherein
the second fusion protein comprises a first fluorescent tag fused to the light-sensitive receptor protein and the self-assembling protein; and
the third fusion protein comprises a second fluorescent tag fused to the low complexity or intrinsically disordered protein region, the cognate partner to the light-sensitive receptor protein, and the PPID.

24. The composition according to claim 13, wherein
the second fusion protein comprises the self-assembling protein and the at least one PPID; and
the third fusion protein comprises the a low complexity or intrinsically disordered protein region (IDR), and the self-assembling protein.

25. The composition according to claim 24, wherein the second fusion protein and/or the third fusion protein further comprises a fluorescent tag.

26. The composition according to claim 25, wherein
the second fusion protein comprises a first fluorescent tag fused to the self-assembling protein and the at least one PPID; and
the third fusion protein comprises a second fluorescent tag fused to the low complexity or intrinsically disordered protein region (IDR) and the self-assembling protein.

27. The composition according to any one of claims 1-12, wherein the at least one additional fusion protein comprises a second fusion protein, the second fusion protein comprising the PPID and the self-assembling protein; and wherein the target protein is a self-interacting protein.

28. The composition according to any one of claims 1-12, wherein the at least one additional fusion protein comprises a second fusion protein, the second fusion protein comprising the PPID, the self-assembling protein, and the low complexity or intrinsically disordered protein region (IDR).

29. The composition according to claim 27 or 28, wherein the second fusion protein further comprises a fluorescent tag.

30. The composition according to any one of claims 10-11, 21-23, 25-26 and 29, wherein the fluorescent protein or fluorescent tag is m-Cherry, Green Fluorescent Protein (GFP), enhanced GFP (EGFP), Cyan Fluorescent Protein (CFP), Yellow Fluorescent Protein (YFP), Red Fluorescent Protein (RFP), Orange Fluorescent Protein (OFP), blue fluorescent protein (BFP), tetracysteine fluorescent motif, or any combination thereof.

31. The composition according to any one of the preceding claims, wherein the PPID is selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

32. The composition according to any one of the preceding claims, wherein the peptide ligand is a peptide capable of binding to a PPID selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

33. The composition according to any one of the preceding claims, wherein the self-assembling protein is ferritin.

34. The composition according to claim 33, wherein the ferritin is a ferritin heavy chain or a ferritin light chain.

35. The composition according to claim 34, wherein the ferritin is a ferritin heavy chain.

36. The composition according to any one of the preceding claims, wherein the intrinsically disordered protein region (IDR) is FUS or FUSn.

37. The composition according to any one of the preceding claims, for use in recruiting the target protein to the at least one additional fusion protein.

38. The composition according to any one of the preceding claims, for use in enhancing a biosynthetic reaction by increasing a local concentration of the target protein, wherein the target protein is one or more enzymes of a metabolic pathway.

39. A method for increasing production of at least one chemical in a cell, the method comprising the steps of expressing, in the cell, a composition according to any one of claims 1-36, under conditions sufficient to form an assembled phase.

40. A method for enhancing a biochemical reaction in a cell, the method comprising the steps of expressing, in the cell, a composition according to any one of claims 1-36, under conditions sufficient to form an assembled phase.

41. A method of treating a condition or disorder in a subject, the method comprising the step of expressing a composition according to any one of claims 1-36 in a cell of the subject under conditions sufficient for the at least one additional fusion protein to form an assembled phase.

42. The method according to claim 41, wherein the condition or disorder is a condition or disorder of a metabolic, signaling, a transcription, a translation, or degradation pathway.

43. A cell comprising the composition according to any one of claims 1-36.

44. The cell according to claim 43, wherein the cell is a human cell, an animal cell, or a yeast cell.

45. A engineered system comprising a plurality of nucleic acids encoding the fusion proteins of any one of claims 1-36.

46. An engineered system comprising
a first nucleic acid construct encoding a target protein fused to a peptide ligand; and
at least one additional nucleic acid construct comprising:
(a) a second nucleic acid construct encoding a self-assembling protein and at least one protein-peptide interaction domain (PPID); or
(b) a second nucleic acid construct encoding a self-assembling protein, and a third nucleic acid construct encoding a low complexity or intrinsically disordered protein region (IDR), wherein either the second nucleic acid construct or third nucleic acid construct further encode at least one PPID;
wherein, when expressed in a cell, the peptide ligand is capable of binding to the at least one PPID.

47. The system according to claim 46, wherein the first nucleic acid construct or the at least one additional nucleic acid construct further comprises a promoter.

48. The system according to claim 46 or 47, wherein the first nucleic acid construct or the at least one additional nucleic acid construct further comprises a sequence encoding a polyadenylation tail.

49. The system according to any one of claims 46-48, wherein the first nucleic acid construct or the at least one additional nucleic acid construct further comprises an origin of replication.

50. The system according to any one of claims 46-49, wherein the first nucleic acid construct or the at least one additional nucleic acid construct comprises a sequence encoding a 5′ untranslated region (5′-UTR).

51. The system according to any one of claims 46-50, wherein the first nucleic acid construct or the at least one additional nucleic acid construct comprises a sequence encoding a 3′ untranslated region (3′-UTR).

52. The system according to any one of claims 46-51, wherein the first construct or the at least one additional construct further comprises a restriction site.

53. The system according to claim 52, wherein the restriction site is a NotI, XhoI, SpeI, EcoRI, BamHI, HinFI, XbaI, NheI, MreI, XmaI, AgeI, BspEI, PacI, PmeI, KpnI, SacI, or AscI restriction enzyme cutting site.

54. The system according to any one of claims 46-53 wherein, when expressed in a cell, said at least one additional fusion protein is configured to form an assembled phase, the assembled phase comprising at least one aggregate.

55. The system according to claim 54, wherein the aggregate comprises phase separated clusters.

56. The system according to claim 55, wherein the peptide ligand binds to the PPID, thereby recruiting the target protein to the phase-separated clusters.

57. The system according to claim 55 or 56, wherein the phase-separated clusters form upon exposure of said at least one additional fusion protein to a stimulus selected from the group consisting of light, temperature, chemicals, and any combination thereof.

58. The system according to any one of claims 46-57, wherein the system is expressed.

59. The system according to claim 58, wherein expression of the system increases production of at least one chemical in the cell as compared with a cell that does not contain the system.

60. The system according to any one of claims 46-59, wherein the target protein encoded by the first fusion protein is an enzyme.

61. The system according to claim 60, wherein the enzyme is an enzyme of a metabolic pathway.

62. The system according to any one of claims 46-59, wherein the target protein encoded by the first fusion protein is a fluorescent protein.

63. The system according to any one of claims 46-62, wherein the at least one additional construct further encodes at least one fluorescent tag.

64. The system according to any one of claims 46-63, comprising a plurality of first constructs.

65. The system according to any one of claims 46-64, wherein the at least one additional nucleic acid construct comprises a second nucleic acid construct encoding the self-assembling protein, and a third nucleic acid construct encoding the low complexity or intrinsically disordered protein region (IDR).

66. The system according to claim 65, wherein
the second nucleic acid construct encodes the self-assembling protein and a light-sensitive receptor protein; and
the third nucleic acid construct encodes the low complexity or intrinsically disordered protein region (IDR), and a cognate partner to the light-sensitive receptor protein.

67. The system according to claim 66, wherein the second nucleic acid construct encodes the PPID.

68. The system according to claim 66, wherein the third nucleic acid construct encodes the PPID.

69. The system according to any one of claims 66-68, wherein the light-sensitive receptor protein is iLID.

70. The system according to any one of claims 66-69, wherein the cognate partner to the light-sensitive receptor protein is sspB.

71. The system according to any one of claims 66-70, wherein the light-sensitive receptor protein is sensitive to at least one visible, ultraviolet (UV) or infrared (IR) wavelength of light.

72. The system according to any one of claims 66-71, wherein, when expressed in a cell, the cognate partner of the light-sensitive receptor protein is configured to bind to the light-sensitive receptor protein when the system is irradiated with at least one wavelength of light.

73. The system according to any one of claims 65-72, wherein the second nucleic acid construct and/or the third nucleic acid construct further encodes a fluorescent tag.

74. The system according to claim 73, wherein
the second nucleic acid construct encodes a first fluorescent tag fused to the light-sensitive receptor protein, the self-assembling protein and the PPID; and
the third nucleic acid construct encodes a second fluorescent tag fused to the low complexity or intrinsically disordered protein region (IDR) and the cognate partner to the light-sensitive receptor protein.

75. The system according to claim 73, wherein
the second nucleic acid construct encodes a first fluorescent tag fused to the light-sensitive receptor protein and the self-assembling protein; and
the third nucleic acid construct encodes a second fluorescent tag fused to the low complexity or intrinsically disordered protein region (IDR), the cognate partner to the light-sensitive receptor protein, and the PPID.

76. The system according to claim 65, wherein
the second nucleic acid construct encodes a self-assembling protein and at least one PPID; and
the third nucleic acid construct encodes a low complexity or intrinsically disordered protein region, and a self-assembling protein.

77. The system according to claim 76, wherein the second nucleic acid construct and/or the third nucleic acid construct further encodes a fluorescent tag.

78. The system according to claim 77, wherein
the second nucleic acid construct encodes a first fluorescent tag fused to the self-assembling protein and the at least one PPID; and
the third nucleic acid construct encodes a second fluorescent tag fused to the low complexity or intrinsically disordered protein region (IDR), and the self-assembling protein.

79. The system according to any one of claims 46-64, wherein the at least one additional nucleic acid construct comprises a second nucleic acid construct, the second nucleic acid construct encoding the PPID and the self-assembling protein; and
wherein the target protein is a self-interacting protein.

80. The system according to any one of claims 46-64, wherein the at least one additional nucleic acid construct comprises a second nucleic acid construct, the second fusion construct encoding the PPID, the self-assembling protein, and the low complexity or intrinsically disordered protein region (IDR).

81. The system according to claim 79 or 80, wherein the second nucleic acid construct further encodes a fluorescent tag.

82. The system according to any one of claims 62-63, 73-75, 77-78, and 81, wherein the fluorescent protein or tag is m-Cherry, Green Fluorescent Protein (GFP), enhanced GFP (EGFP), Cyan Fluorescent Protein (CFP), Yellow Fluorescent Protein (YFP), Red Fluorescent Protein (RFP), Orange Fluorescent Protein (OFP), blue fluorescent protein (BFP), tetracysteine fluorescent motif, or any combination thereof.

83. The system according to any one of claims 46-82, wherein the PPID is selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

84. The system according to any one of claims 46-82, wherein the peptide ligand is capable of binding to a PPID selected from the group consisting of a Src homonology-2 (SH2) domain, a Src homology-3 (SH3) domain, a ALFA-Nb domain, a PSD95/DlgA/Zo-1 (PDZ) domain, a WW domain, a GTPase Binding Domain (GBD), a leucine zipper domain, a forkhead associated (FHA) domain, a 14-3-3 domain, a death domain, a caspase recruitment domain (CARD), a bromodomain, a chromatin organization modifier, a shadow chromo domain, an F-box domain, a HECT domain, a RING finger domain, a sterile alpha motif (SAM) domain, a glycine-tyrosine-phenylalanine (GYF) domain, a soluble NSF attachment protein (SNAP) domain, a VHS domain, an ANK repeat, an armadillo repeat, a WD40 repeat, an MH2 domain, a calponin homology domain, a Dbl homology domain, a gelsolin homology domain, a phox and Bem1 (PB1) domain, a SOCS box, an RGS domain, a Toll/IL-1 receptor domain, a tetratricopeptide repeat, a TRAF domain, a Bcl-2 homology domain, a coiled-coil domain, a bZIP domain and a phosphotyrosine-binding domain (PTB).

85. The system according to any one of claims 46-83, wherein the self-assembling protein is ferritin.

86. The system according to claim 85, wherein the ferritin is a ferritin heavy chain or a ferritin light chain.

87. The system according to claim 86, wherein the ferritin is a ferritin heavy chain.

88. The system according to any one of claims 46-87, wherein the intrinsically disordered protein region (IDR) is FUS or FUSn.

89. The system according to any one of claims 46-88, for use in recruiting the target protein to the at least one additional fusion protein.

90. The system according to any one of claims 46-88, for use in enhancing a biosynthetic reaction by increasing a local concentration of the target protein, wherein the target protein is one or more enzymes of a metabolic pathway.

91. A method of forming an assembled phase, comprising:
expressing, within a cell, the composition according to any one of claims 1-36; and
allowing the composition to undergo phase separation into at least one assembled phase within the living cell.

92. The method of claim 91, wherein the at least one assembled phase comprises phase-separated clusters.

93. A method for screening for protein-protein interactions, the method comprising:
expressing, in a cell, a first composition according to any one of claims 1-36 comprising a first target protein, and a second composition according to any one of claims 1-36 comprising a second target protein, under conditions sufficient to form an assembled phase; and
measuring colocalization of the first and the second target proteins in the cell.

94. A method of screening for compounds capable of modulating a protein-protein interaction, the method comprising:
expressing, in a cell, a first composition according to any one of claims 1-36 comprising a first target protein, and a second composition according to any one of claims 1-36 comprising a second target protein, under conditions sufficient to form an assembled phase;
contacting the cell with a test compound;
measuring colocalization of the first and the second target proteins in the cell in the presence of the test compound; and
comparing colocalization of the first and the second target proteins in the presence of the test compound to a reference sample measured in the absence of the test compound;
wherein a change in the colocalization of the first and the second target proteins in the presence of the test compound as compared with the reference sample is indicative of the ability of the test compound to modulate said protein-protein interaction.

95. The method according to claim 93 or 94, wherein measuring colocalization of the first and the second target proteins comprises detecting location of the first and the second target proteins in the cell.

96. The method according to claim 95, wherein detecting comprises imaging.

97. The method according to claim 95 or 96, wherein detecting location of the first and the second target proteins in the cell comprises detecting a signal.

98. The method according to claim 97, wherein the signal is an electronic signal or an electromagnetic signal.

99. The method according to claim 97, wherein the signal is optically detectable.

100. The method according to claim 99, wherein the optically detectable signal is a fluorescence signal or a luminescence signal.

101. The method according to claim 99, wherein the optically detectable signal is a small-molecule dye, a fluorescent molecule or protein, a quantum dot, a colorimetric reagent, a chromogenic molecule or protein, a Raman label, a chromophore, or a combination thereof.

102. The method according to claim 93 or 94, wherein measuring colocalization of the first and the second target proteins comprises determining presence or amount of a compound in a biological pathway.

103. A method for screening for protein-nucleic acid interactions, the method comprising:
expressing, in a cell, a composition according to any one of claims 1-36 comprising a target protein, under conditions sufficient to form an assembled phase; and
measuring binding of a nucleic acid to the target protein in the cell.

104. A method of screening for compounds capable of modulating a protein-nucleic acid interaction, the method comprising:
expressing, in a cell, a composition according to any one of claims 1-36 comprising a target protein, under conditions sufficient to form an assembled phase;
contacting the cell with a test compound;
measuring binding of a nucleic acid to the target protein in the cell in the presence of the test compound; and
comparing binding of the nucleic acid to the target protein in the presence of the test compound to a reference sample measured in the absence of the test compound;
wherein a change in the binding of the nucleic acid to the target protein in the presence of the test compound as compared with the reference sample is indicative of the ability of the test compound to modulate protein-nucleic acid interaction.

105. The method according to claim 103 or 104, wherein measuring binding of the nucleic acid to the target protein comprises detecting a signal from the nucleic acid.

106. The method according to claim 105, wherein measuring further comprises, prior to detecting, staining the nucleic acid or binding the nucleic acid with a detectable probe.

107. The method according to claim 105 or 106, wherein the signal is a fluorescence signal or a luminescence signal.

108. The method according to claim 103 or 104, wherein measuring binding of the nucleic acid to the target protein comprises determining presence or amount of a compound in a biological pathway.

109. The method according to any one of claims 104-108, wherein the compound disrupts binding of the target protein to the nucleic acid.

110. The method according to any one of claims 104-108, wherein the compound enhances binding of the target protein to the nucleic acid.

111. A method of screening for a compound capable of modulating a target protein, the method comprising:
expressing, in a cell, a composition according to any one of claims 1-36, under conditions sufficient to form an assembled phase;
contacting the cell with a test compound;
measuring a biological parameter of the target protein; and
comparing said biological parameter to a reference sample measured in the absence of the test compound;
wherein a change in the biological parameter in the presence of the test compound as compared with the reference sample is indicative of the ability of the test compound to modulate said target protein.

112. The method of claim 111, wherein the biological parameter is enzymatic activity, metabolism, signaling, transcription, translation, degradation, a post-translational modification, or presence or amount of a compound in a biological pathway.

113. The method of claim 111 or 112, wherein modulating the target protein comprises inhibiting the target protein.

114. The method of claim 111 or 112, wherein modulating the target protein comprises activating or increasing the amount of the target protein.

115. The method of claim 111 or 112, further comprising administering to a subject the test compound to treat a condition.


SEQUENCE LISTING (3 sequences)

SEQ ID NO: 1 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 2 - protein, 724 aa, Homo sapiens
MGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESSDTQGPKVTYIPPPPPEDEDSIFAHYQTGINFDKYDTILVEVSGHDAPPAILTFEEANLCQTLNNNIAKAGYTKLTPVQKYSIPIILAGRDLMACAQTGSGKTAAFLLPILAHMMHDGITASRFKELQEPECIIVAPTRELVNQIYLEARKFSFGTCVRAVVIYGGTQLGHSIRQIVQGCNILCATPGRLMDIIGKEKIGLKQIKYLVLDEADRMLDMGFGPEMKKLISCPGMPSKEQRQTLMFSATFPEEIQRLAAEFLKSNYLFVAVGQVGGACRDVQQTVLQVGQFSKREKLVEILRNIGDERTMVFVETKKKADFIATFLCQEKISTTSIHGDREQREREQALGDFRFGKCPVLVATSVAARGLDIENVQHVINFDLPSTIDEYVHRIGRTGRCGNTGRAISFFDLESDNHLAQPLVKVLTDAQQDVPAWLEEIAFSTYIPGFSGSTRGNVFASVDTRKGKSTLNTAGFSSSQAPNPVDDESWD

SEQ ID NO: 3 - protein, 320 aa, Homo sapiens
MSKSASPKEPEQLRKLFIGGLSFETTDESLRSHFEQWGTLTDCVVMRDPNTKRSRGFGFVTYATVEEVDAAMNTTPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVKGHNCEVRKALPKQEMASASSSQRGRRGSGNFGGGRGDGFGGNDNFGRGGNFSGRGGFGGSCGGGGYGGSGDGYNGFGNDGSNFGGGGSYNDFGNYNNQSSNFGPMKGGNFGGRSSGPYGGGGQYFAKPQNQGGYGVSSSSSSYGSGRRF